SEQUENCE LISTING (16 sequences)

SEQ ID NO: 1 - DNA, 22 bp, Artificial Sequence
  Description of Artificial Sequence Synthetic primer W1160
caccgaaatgtttttctctacg

SEQ ID NO: 2 - DNA, 20 bp, Artificial Sequence
  Description of Artificial Sequence Synthetic primer W1161
ctccttggtggttttgttga

SEQ ID NO: 3 - DNA, 45 bp, Artificial Sequence
  Description of Artificial Sequence Synthetic primer W1443
tcaagagacatggcaggcagaggtggaagaggactcgtagctgcg

SEQ ID NO: 4 - DNA, 45 bp, Artificial Sequence
  Description of Artificial Sequence Synthetic primer W1444
cgcagctacgagtcctcttccacctctgcctgccatgtctcttga

SEQ ID NO: 5 - DNA, 33 bp, Artificial Sequence
  Description of Artificial Sequence Synthetic primer W1445
ggactcgtagctgcgaggacgatggctgctaac

SEQ ID NO: 6 - DNA, 33 bp, Artificial Sequence
  Description of Artificial Sequence Synthetic primer W1446
gttagcagccatcgtcctcgcagctacgagtcc

SEQ ID NO: 7 - DNA, 40 bp, Artificial Sequence
  Description of Artificial Sequence Synthetic primer W1447
gacgatggctgctaacagggacagagacaaggacaagaag

SEQ ID NO: 8 - DNA, 40 bp, Artificial Sequence
  Description of Artificial Sequence Synthetic primer W1448
cttcttgtccttgtctctgtccctgttagcagccatcgtc

SEQ ID NO: 9 - protein, 139 aa, Oryza sativa
MAGKGGKGLLAAKTTAAKAAADKDKDRKKAPVSRSSRAGIQFPVGRIHRQLKGRVSANGRVGATAAVYTAAILEYLTAEVLELAGNASKDLKVKRITPRHLQLAIRGDEELDTLIKGTIAGGGVIPHIHKSLINKTAKE

SEQ ID NO: 10 - protein, 138 aa, Oryza sativa
MAGKGGKGLLAAKTTAAKSADKDKDKKKAPVSRSSRAGLQFPVGRIHRQLKSRASAHGRVGATAAVYSAAILEYLTAEVLELAGNASKDLKVKRITPRHLQLAIRGDEELDTLIKGTIAGGGVIPHIHKSLINKTSKE

SEQ ID NO: 11 - protein, 137 aa, Oryza sativa
MAGKGGKGLLAAKTTAAKSAEKDKGKKAPVSRSSRAGLQFPVGRIHRQLKQRTQANGRVGATAAVYSAAILEYLTAEVLELAGNASKDLKVKRITPRHLQLAIRGDEELDTLIKGTIAGGGVIPHIHKSLINKSSKE

SEQ ID NO: 12 - protein, 134 aa, Arabidopsis thaliana
MSGKGAKGLIMGKPSGSDKDKDKKKPITRSSRAGLQFPVGRVHRLLKTRSTAHGRVGATAAVYTAAILEYLTAEVLELAGNASKDLKVKRISPRHLQLAIRGDEELDTLIKGTIAGGGVIPHIHKSLINKSAKE

SEQ ID NO: 13 - protein, 136 aa, Arabidopsis thaliana
MAGKGGKGLLAAKTTAAAANKDSVKKKSISRSSRAGIQFPVGRIHRQLKQRVSAHGRVGATAAVYTASILEYLTAEVLELAGNASKDLKVKRITPRHLQLAIRGDEELDTLIKGTIAGGGVIPHIHKSLVNKVTKD

SEQ ID NO: 14 - protein, 136 aa, Arabidopsis thaliana
MAGKGGKGLVAAKTMAANKDKDKDKKKPISRSARAGIQFPVGRIHRQLKTRVSAHGRVGATAAVYTASILEYLTAEVLELAGNASKDLKVKRITPRHLQLAIRGDEELDTLIKGTIAGGGVIPHIHKSLINKTTKE

SEQ ID NO: 15 - protein, 132 aa, Zea mays
MAGKGGGKSLVAAKIAAGMDKDKKSPVNRSARAGLHFPVGRIHRHLKDSAQAGERVGATAAVYAAAVLEYLTAEIMELAGNACRDLKVKRITPRHLQLAVRGDEELDTLIKATIAGGGVIPHIHRELIDKAA

SEQ ID NO: 16 - protein, 138 aa, Zea mays
MAGKGGKGLLAAKTTAAKSTDKDKDRKKAPVSRSSRAGLQFPVGRIHRQLKSRASAHGRVGATAAVYSAAILEYLTAEVLELAGNASKDLKVKRITPRHLQLAIRGDEELDTLIKGTIAGGGVIPHIHKSLINKTAKE

(19) United States
(12) Patent Application Publication
(10) Pub. No.: US 2013/0239252 A1
(43) Pub. Date: Sep. 12, 2013

(54) Methods and Compositions for Altering Temperature Sensing in Eukaryotic Organisms

(71) Applicants: Philip Wigge, Norwich (GB); Vinod Kumar, Norwich (GB)

(72) Inventors: Philip Wigge, Norwich (GB); Vinod Kumar, Norwich (GB)

(73) Assignee: Plant Bioscience Limited, Norwich (GB)

(21) Appl. No.: 13/520,967

(22) PCT Filed: Jun. 8, 2010
(86) PCT No.: PCT/GB10/50956

§ 371 Date: May 16, 2013

(30) Foreign Application Priority Data
Jan. 7, 2010 (GB) 1000184.0

(51) Int. Cl. C12N 15/82

(52) U.S. Cl. CPC C12N 15/8261
USPC 800/278; 530/370; 800/298; 530/376; 530/375; 530/372

(57) ABSTRACT

H2A.Z containing nucleosomes mediate the thermosensory response in plants and other eukaryotes and modifications to H2A.Z alter such responses.

### FIELD OF THE INVENTION

H2A.Z-containing nucleosomes mediate the thermosensory response in eukaryotes and modifications to H2A.Z alter this response.

### BACKGROUND OF THE INVENTION

Sessile organisms, such as plants, continually sense environmental conditions to adapt their growth and development. Temperature varies both diurnally, which is important for entraining the clock (Michael et al., 2008; Salome and McClung, 2005), as well as seasonally, providing information for the timing of reproduction (Heggie and Halliday, 2005; Samach and Wigge, 2005; Sung and Amasino, 2005). Extremes of temperature represent a significant stress for plants, and are a major factor limiting global plant distribution (Mittler, 2006). The sensitivity of plants to small changes in temperature is highlighted by significant changes in flowering time (Fitter and Fitter, 2002) and distributions of wild-plants that have occurred in the last 100 years due to climate change (Kelly and Goulden, 2008; Lenoir et al., 2008; Willis et al., 2008). Projected increases in mean global temperature, as well as extremes of temperature in the next 100 years, are significantly larger than what has occurred so far, suggesting significant future disruption to wild plants and crops.

Plants must constantly respond to changes in the environment whilst maintaining developmental and growth processes if they are to survive into the next generation. A complex network of signals from temperature and light must correctly converge to achieve successful development, through vegetative to reproductive growth. Temperature can be thought of as an environmental factor that provides both ‘inductive’ and ‘maintenance’ signals in development. It can stimulate developmental processes such as seed dormancy release, germination, vernalization and flowering. Thus, being able to control how a plant perceives temperature is desirable. The responses of organisms to temperature, particularly in crop plants, are often detrimental, and the targeted alteration of these responses may improve the yield, quality and predictability of crops. For example grain-fill in wheat and rice is particularly sensitive to high temperatures. Preventing a heat shock response in the developing grain would protect grain composition and quantity from being perturbed by a high temperature stress response. High temperature stress responses have been selected for in evolution and breeding in order to maximise embryo survival, but these responses are detrimental to grain quality. This is already a major cause of reduced wheat and rice yields during hot summers, and these problems will be exacerbated by future climate change.

Additionally many crops flower or bolt prematurely when exposed to high or low temperatures (for example sugar beet, lettuce, onions, cabbage and broccoli), which is a major cause of crop loss. Specifically controlling the flowering responses to temperature in such crops is desirable. Furthermore, reducing temperature sensitivity may enable a more uniform development within a crop, which is important, since it enables farmers to schedule harvests reliably and optimises crop yield and quality. Additionally, many harmful eukaryotic micro-organisms use temperature perception as a signal to induce pathogenesis, for example Histoplasma capsulatum and Candida albicans (Nguyen and Sil, 2008). The ability to selectively perturb temperature perception in these organisms would provide means to prevent pathogenesis.

Temperature sensing in eukaryotes is a complex process that is not yet fully understood. In plants, factors that are involved in temperature sensing include calcium signalling and low temperature is known to change membrane fluidity.

Genetic information within the eukaryotic cell nucleus is organized in a highly conserved structural polymer, the chromatin, which supports and controls crucial functions of the genome. The basic unit of chromatin is the nucleosome, which contains DNA wrapped around a histone octamer. Core histones H2A, H2B, H3, and H4 have a highly structured core domain involved in intranucleosomal histone-histone interactions and unstructured N- and C-terminal tails that extend outside of the nucleosomal core, and they interact with other nucleosomes and with nonhistone proteins. These tails are subject to a number of post-translational modifications, such as acetylation, methylation, phosphorylation, and ubiquitination. The organization of DNA into chromatin is inhibitory to all the processes that need DNA as a template, such as replication, transcription, recombination, and repair. There are a number of mechanisms by which chromatin structure and composition can be manipulated to facilitate these events. These include post-translational modifications of histones. These modifications can alter DNA-histone interactions within and between nucleosomes and, thus, affect higher-order chromatin structures. Another mechanism of modulating chromatin structure is carried out by multisubunit ATP-dependent chromatin remodelling complexes.

In addition to genes encoding the core histone H2A, the eukaryotic genome contains genes referred to as H2A variants, which have a different protein sequences from core H2A. Histone variants play critical roles in chromatin structure and function and are implicated in a variety of cellular activities, such as regulation of transcription, DNA repair, chromosome X inactivation, and heterochromatin formation. H2A.Z variants are highly conserved from Saccharomyces cerevisiae to human. H2A.Z variants have also been identified in plants.

In summary, being able to control temperature regulated responses in plants is of major importance in controlling yield, in particular in view of climate change.

Moreover, being able to regulate temperature responses in other eukaryotic organisms, such as yeast is also desirable.

We have identified methods and compositions which are aimed at meeting this need.

### SUMMARY OF THE INVENTION

We have shown that temperature responses in eukaryotic cells are mediated by the presence of H2A.Z in the nucleosome, and we disclose herein ways in which temperature perception may be specifically altered to optimise responses of plants, yeast, fungi or other eukaryotic organisms. We have found that H2A.Z-containing nucleosomes represent the major node of regulation of responses to temperature and to temperature changes in plants.

We have demonstrated that the gene transcription of temperature responsive genes is coordinated by H2A.Z-containing nucleosomes in plants. We have shown that the presence of H2A.Z in the nucleosome decreases with an increase in temperature, and that this effect is independent of transcription. Without H2A.Z being present in the nucleosome, plants display a constitutive warm temperature response, developmentally and transcriptionally. Thus, the invention relates to methods for altering temperature sensing in a eukaryotic organism and to producing a eukaryotic organism with altered temperature sensing. The invention also relates to methods for altering thermosensory responses in a eukaryotic organism and to producing a eukaryotic organism with altered thermosensory responses.

In a first aspect, the invention relates to a method for altering temperature sensing in a eukaryotic organism comprising modifying the presence or state of H2A.Z in the nucleosome.

In another aspect, the invention relates to a method for altering thermosensory responses in a eukaryotic organism said method comprising decreasing the level of H2A.Z in said organism or altering post-translational modification of H2A.Z or a combination thereof.

In a third aspect, the invention relates to a method for inhibiting the response of a eukaryotic organism to an increase in ambient temperature comprising increasing the presence of H2A.Z in the nucleosome or preventing release of H2A.Z from the nucleosome, altering the state of H2A.Z, such as post-translational modification of H2A.Z, or inhibiting disassembly of the H2A.Z from the nucleosome.

In a fourth aspect, the invention relates to a method for eliciting a warm temperature response in a eukaryotic organism comprising inhibiting or decreasing the presence H2A.Z in the nucleosome.

In another aspect, the invention relates to a method for eliciting a cold temperature response comprising inhibiting or decreasing the eviction of H2A.Z from the nucleosome.

In a further aspect, the invention relates to a method for producing a eukaryotic organism that shows a constitutive warm temperature response comprising inhibiting or decreasing the presence H2A.Z in the nucleosome.

The invention also relates to a method for producing a eukaryotic organism that shows a constitutive cold temperature response comprising inhibiting or decreasing the eviction of H2A.Z from the nucleosome.

Other objects and advantages of this invention will be appreciated from a review of the complete disclosure provided herein and the appended claims.

### BRIEF DESCRIPTION OF THE DRAWINGS

FIG. 1. HSP70 expression is an output of the ambient temperature sensing pathway.

(A) Transcript profiling experiments show a robust transcriptional response to changes in ambient temperature.

(B) HSP70 (black line, marked with an arrow) is induced strongly with increasing ambient temperature. In contrast, the other members of the HSP70 family genes (gray) require a heat stress to be up-regulated.

(C) HSP70 (black line, marked with an arrow) has a uniform linear expression pattern at various constant growth temperatures, in contrast to the rest of the HSP70 family (gray). HSP70 is therefore an excellent output of the thermosensory pathway over a wide temperature range.

(D) HSP70::LUC in Arabidopsis mimics the endogenous HSP70 expression pattern in response to temperature. LUC transcript analysis and live luciferase imaging of plants shifted to 17° C., 22° C. and 27° C. for two hours from 12° C. show a temperature dependent LUC expression and luciferase activity. LUC transcript levels were normalised to UBQ10.

(E) Identification of entr1 through a forward genetic screen as a mutant with enhanced temperature response at the seedling stage (upper panel), this phenotype is present throughout the life cycle. Lower panel shows the HSP70::LUC expression in adult wild-type and entr1 plants.

(F) Transcript profiling analysis showing ARP6 gene expression in WT and entr1. ARP6 transcripts were absent in entr1. The bar represents normalised expression levels from the microarray experiment.

(G) Complementation of entr1: a binary construct containing the ARP6 genomic fragment including the native promoter and coding regions (P_{ARP6}::ARP6) completely rescued the HSP70::LUC as well as the developmental phenotypes of entr1.

FIG. 2. arp6-10 displays developmental and architectural phenotypes of warm grown plants.

(A-D) arp6-10 flowers significantly earlier than wild-type under long-day (A, B) and short day conditions (C, D) at 22° C. When grown in short days at 27° C. (E, F), arp6-10 shows a very strong thermal induction of flowering. In comparison, wild-type flowering time is similar to arp6-10 at 22° C. (x-axis shows the number of rosette leaves at the time of flowering, y-axis shows the corresponding number of plants). (G, H) In addition to the flowering time phenotypes, arp6-10 displays all the architectural responses of wild-type plants grown at high temperature. arp6-10 seedlings at 17° C., show hypocotyl (G) and petiole elongation (H) that is equivalent to the wild-type phenotype at higher temperatures. (Petiole elongation was analysed on the first four true leaves of 20-day-old wild-type and arp6-10 seedlings). See also FIG. 8.

FIG. 3. The temperature transcriptome is globally mis-regulated in arp6-10.

Genes that are 2 fold up- (A) or down-regulated (B) in wild-type seedling within 2 hours of shifting to 27° C. are constitutively repressed or activated in the arp6-10 background. Similarly genes that are 2 fold up- (C) or down-regulated (D) in arp6-10 compared to the wild-type at 12° C. (0 h) were down- or up-regulated in the wild-type upon shift to 27° C. Median and mean values are represented respectively by the solid and dotted horizontal lines. 5th and 95th percentiles represent outlier values (dots).

(E) Comparison of arp6-10 induced genome-wide transcriptional changes with responses to increasing temperature from 12° C. to 27° C. in wild-type. Whole genome scatter plot analysis of expression log ratios. x-axis: comparison of wild-type after 24 h at 27° C. with seedlings kept at 12° C. y-axis: comparison of arp6-10 grown at 12° C. with wild-type at 12° C. The arp6-10 mutation accounts for nearly half (R²=0.466) of the changes in the transcriptome in response to an increase in ambient temperature.

FIG. 4. H2A.Z occupancy varies as a function of ambient temperature

(A) HTA11:GFP expressed under the native promoter is functional and complements the hta9 hta11 double mutant. The hta9 hta11 mutant has an extended hypocotyl compared to Col-0 at 21° C. This phenotype was completely rescued in three independent lines expressing HTA11:GFP under the native promoter (left panel). HTA11:GFP also complements the elevated HSP70 expression in the hta9 hta11 double mutant (right panel).

(B) Enrichment of H2A.Z at the HSP70 promoter was analysed using HTA11:GFP. Formaldehyde cross-linked chromatin from seven day old seedlings grown at 17° C. (open circles) or after 2 hours of incubation at 27° C. (closed circles) were digested with MNase and were used for chromatin immunopurification (ChIP) analyses. H2A.Z immuno-complex associated DNA fragments were quantitatively analysed using tiling oligonucleotides (See FIG. 4B). A region corresponding to the +1 nucleosome is enriched in H2A.Z where as the −1 nucleosome has a much reduced H2A.Z occupancy (inset bar graph, 4A). x-axis: the nucleotide positions across HSP70 gene with respect to TSS. y-axis: H2A.Z occupancy as a fraction of undigested input chromatin. Values are mean±SD from one representative experiment. Bar graph in the inset shows the −1 nucleosome in detail as represented by the amplicon centred on −246 bp.

(C) ChIP analysis of cross-linked H3 on seedlings treated similarly as above shows the well positioned nucleosomes of HSP70 promoter. On temperature shift to 27° C., H3 occupancy changed at the +1 nucleosome where H2A.Z occupancy is high. The relative change in H3 occupancy in response to temperature shift was lower than that of H2A.Z.

(D) A gypsy like transposon gene (At4g07700) acts as a control since it has positioned nucleosomes devoid of H2A.Z and it is transcriptionally inactive and unresponsive to temperature changes. ChIP analysis revealed no change in histone H3 occupancy at both 17° C. and 27° C., indicating that H2A.Z confers temperature specific responsiveness to nucleosomes. As expected, H2A.Z occupancy was absent as measured by ChIP. See also FIG. 9.

FIG. 5. H2A.Z occupancy dynamics are independent of the transcriptional response.

(A-I) Genes with diverse transcriptional response were analysed for H2A.Z occupancy in response to temperature shift. H2A.Z occupancy is primarily determined by temperature, independent of the specific transcriptional response. This result indicates that H2A.Z containing nucleosomes confer temperature information on chromatin, which could be interpreted according to the respective regulatory environment for appropriate gene expression. Upper panels show the transcriptional response upon shift to 27° C. 0 h represents sample from plants grown at 12° C.; 2 h and 24 h are after shift to 27° C. H2A.Z occupancy dynamics upon shift to 27° C. for 2 h is shown in the lower panel.

FIG. 6. Chromatin architecture responds dynamically to changes in ambient temperature.

(A) Chromatin architecture dynamics at HSP70 in response to temperature in wild-type. Chromatin from seven day old seedlings growing at 17° C. (open circle), 22° C. (closed circle) and 27° C. (square) was MNase treated and analysed by Q-PCR with tiled oligos. The +1 nucleosome of HSP70 exhibits a robust response to increasing ambient temperature, with nucleosome occupancy and temperature being negatively correlated.

(B) arp6 shows a constitutive temperature response in terms of chromatin architecture as measured by MNase accessibility. +1 nucleosome occupancy in arp6 is consistently lower, even at 17° C.

(C) Analysis of nucleosome occupancy dynamics in response to temperature at At4g07700 reveals that consistent with the H3 data, nucleosomes at this locus are non-responsive to changes in ambient temperature.

ChIP analysis for RNA Pol II at HSP70 in wild-type (closed circle) and arp6 (open circle) grown at 17° C. (D) or after 2 h of incubation at 27° C. (E). x-axis represents nucleotide positions on HSP70 with respect to TSS; y-axis represents fraction of input chromatin immunopurified using RNA Pol II antibody.

(F) Schematic representation of HSP70 +1 and −1 nucleosomes showing the Hph I sites. Grey ovals represent nucleosomes, oligo positions are indicated by arrows. (G-H) Temperature-dependent nucleosomal DNA accessibility assay for Hph I enzyme in wild-type (G) and arp6-10 (H). Nucleosomes containing H2A nucleosomes (−1 in wild-type and both −1 and +1 in arp6-10) show constitutive accessibility of restriction sites, whereas H2A.Z containing nucleosomes occlude access. Relative amounts of protected input nucleosomal DNA was calculated and was normalised against the +1 nucleosome of At4g07700 where Hph I does not cut. See also FIG. 10.

FIG. 7. H2A.Z containing nucleosomes can modulate transcription in a temperature dependent manner.

At lower temperature, H2A.Z nucleosomes have a high level of occupancy. H2A.Z nucleosomes may prevent transcription, either by acting as a physical block to the progression of RNA Pol II, or by occluding gene specific cis-elements from activating transcription factors. For genes that are specifically expressed at low temperature, H2A.Z occupancy may prevent the binding of repressors or antagonise DNA methylation. A chromatin-remodelling complex may relieve the nucleosomal repression when appropriate components and conditions exist. At higher temperature, H2A.Z nucleosome occupancy declines. This leads to increased expression of genes like HSP70, where transcription is limited by H2A.Z occupancy. For genes whose expression is decreased at higher temperature, this loss of H2A.Z may facilitate repressor binding. Conventional H2A nucleosomes are depicted in light gray ovals and the degree of H2A.Z occupancy is depicted in darker gray.

FIG. 8. Temperature induced architectural phenotypes are constitutive in arp6-10

(A) Hypocotyl elongation was quantified from 15-day-old seedlings of WT and arp6-10 grown at respective temperatures were quantified. (B-D) Petiole elongation in first four leaves of 20-day-old seedlings of WT and arp6-10 grown at 17° C. (B), 22° C. (C) and 27° C. (C) were quantified.

FIG. 9. HTA11:GFP fusion protein is localized and functions as the endogenous protein

Confocal microscopy reveals the nuclear distribution of HTA11:GFP in the nucleus and exclusion from the heterochromatic regions (A) consistent with the reported pattern of the endogenous protein (Deal et al., 2007, Zilberman et al., 2008). (B) Visualisation of an anaphase cell shows the incorporation of HTA11:GFP into chromosomes. (C) Chromatin immunoprecipitation analysis of MNase digested chromatin from arp6-10 shows the drastic reduction of H2A.Z deposition into +1 nucleosome of HSP70. H2A.Z occupancy profile of wild type Col-0 is shown as dotted line for reference. Insert shows the arp6-10 profile alone. (D) Chromatin from 17° C. grown seedlings as well as, from those shifted to 27° C. for 2 h were digested with MNase and were used for ChIP experiments using antibody against H2A.Z showing temperature responsive H2A.Z dynamics. These results further validate the HTA11:GFP protein fusion and our ChIP results using the same. (E) ChIP for H2A.Z occupancy at the TSS proximal region of FT, a key regulator of the thermal induction of flowering. Consistent with the other results we found that H2A.Z occupancy is at this locus is a function of ambient temperature temperature.

FIG. 10. HTZ1 regulates by temperature regulon in yeast.

Expression of genes assigned to be up- and down-regulated in the htz1Δ background (y-axis) (Meneghini et al., 2003) was compared with gene expression on shifting from 29° C. to 33° C. (x-axis) (Gasch et al., 2000). There is a strong correlation: genes constitutively down-regulated in the htz1Δ background tend to be those genes whose expression is inhibited on increasing temperature, while those genes whose expression goes up at 33° C. tend to be more highly expressed in the htz1Δ mutant at 30° C. These results are consistent with a role for Htz1 containing nucleosomes to provide thermal information for regulating the temperature transcriptome. Temperature induced loss of Htz1 from chromatin at higher temperatures may contribute to the regulation of temperature responsive genes (R² 0.30, P<0.001).

FIG. 11. Peptide sequences encoding H2A.Z in Arabidopsis, rice and maize and sequence alignment

FIG. 12. Flowering data from HAC miRNA lines

Over-expression of an artificial microRNA targeting the HAC genes in Arabidopsis results in delayed flowering. Two representative stable transgenic lines #5 and #2 are shown with the wildtype at two different time points, one at the time the wildtype flowers (A) and many weeks later (B) to show the extend of delayed flowering. The bar graph (C) shows the flowering data in terms of leaf number at the time of flowering. Dark bars represent rosette leaves and grey bar represent cauline leaves.

### DETAILED DISCLOSURE OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The practice of the present invention will employ, unless otherwise indicated, conventional techniques of botany, microbiology, tissue culture, molecular biology, chemistry, biochemistry and recombinant DNA technology, which are within the skill of the art. Such techniques are explained fully in the literature.

Plants are highly sensitive to temperature and can perceive a difference of as little as 1° C. How temperature is sensed and integrated in development is largely unknown. We have found that the presence or absence of the alternative histone H2A.Z in the nucleosome is essential for how a plant senses temperature and temperature changes. Surprisingly, we have found that the presence or absence of the alternative histone H2A.Z in the nucleosome is an essential central point of regulation in the perception of temperature and thus the thermosensory response, which includes regulation of the expression of a large number of temperature dependent genes (the temperature transcriptome). Thus, although temperature sensing and eliciting temperature-dependent responses is known to be a complex biological process, the inventors have shown that a central single regulator in this mechanism is H2A.Z. Moreover, the inventors have shown that by modifying the presence or absence of H2A.Z in the nucleosome, it is possible to regulate the ways in which a plant perceives temperature and thus to alter developmental response that are dependent on temperature. It is therefore possible to alter a large variety of temperature dependent responses, including developmental responses in plants such as flowering, hypocotyl elongation and petiole growth, by targeting this single regulator.

Thus, the methods of the invention relate to altering temperature sensing in a eukaryotic organism, for example in a plant, and thus modifying thermosensory responses by altering the presence of H2A.Z in the nucleosome.

As shown herein, the presence of H2A.Z in the nucleosome is rate limiting for the expression of genes that are dependent on increasing temperature. Thus, in a wild type organism, H2A.Z is present in the nucleosome at cold temperatures. H2A.Z responds to an increase in the ambient temperature and is released from the nucleosome. This release induces the expression of temperature dependent genes that are expressed at higher temperatures, whilst cold temperature dependent genes are no longer expressed. H2A.Z thus functions as a temperature sensor and the presence or absence of this regulator determines gene expression which in turn regulates developmental responses that are normally induced by changes in temperature. As discussed in the examples, when H2A.Z deposition is prevented, plants display a constitutive warm temperature response even in cold temperature, i.e. a response that the plant would normally display in warm temperature.

As shown herein, genotypes deficient in incorporating H2A.Z into nucleosomes phenocopy warm grown plants even when grown at lower temperatures. As a skilled person will appreciate responses to changes in temperature vary between different plants, depending on the specific temperature threshold of the plant. However, generally speaking, by warm grown plants is meant plants grown at a temperature of about 20° C. to about 24° C., for example about 22° C. or above for Arabidopsis. By cold temperature is meant plants gown at a temperature of about 15° C. to about 19° C., for example about 17° C. or below for Arabidopsis. Other plants have different temperature optima.

Genotypes deficient in incorporating H2A.Z into nucleosomes also show altered gene expression and the gene expression profile is that typically shown by plants grown in warm temperature conditions. Thus, nucleosomes containing H2A.Z display distinct responses to temperature in vivo, independently of transcription. Using purified nucleosomes, we are able to show that H2A.Z confers distinct DNA unwrapping properties on nucleosomes, indicating a direct mechanism for the perception of temperature through DNA-nucleosome fluctuations. Our results show that H2A.Z-containing nucleosomes provide thermosensory information that is used to coordinate developmental responses and the ambient temperature transcriptome. We observe the same effect in budding yeast, indicating this is an evolutionarily conserved mechanism.

In a first aspect, the invention relates to a method for altering temperature sensing in a eukaryotic organism comprising modifying the presence of H2A.Z in the nucleosome.

In another aspect, the invention relates to a method for altering thermosensory responses in a eukaryotic organism said method comprising decreasing the level of H2A.Z in said organism or altering post-translational modification of H2A.Z or a combination thereof.

The eukaryotic organism according to the different methods of the invention may be a plant, a yeast, a fungus, an invertebrate or a vertebrate. In one embodiment, the organism is a plant. The organism may also be an isolated part of a plant, such as a plant cell, for example a plant cell culture or cell line. Thus, in one aspect, the invention relates to a method for altering temperature sensing in a plant, a plant cell culture or cell line comprising modifying the presence of H2A.Z in the nucleosome and to a method for altering thermosensory responses in a plant said method comprising decreasing the level of H2A.Z in said organism or altering post-translational modification of H2A.Z or a combination thereof.

Where the organism is a plant, it may be a moncot or a dicot.

A dicot plant may be selected from the families including, but not limited to Asteraceae, Brassicaceae (eg Brassica napus), Chenopodiaceae, Cucurbitaceae, Leguminosae (Caesalpiniaceae, Aesalpiniaceae Mimosaceae, Papilionaceae or Fabaceae), Malvaceae, Rosaceae or Solanaceae. For example, the plant may be selected from lettuce, sunflower, Arabidopsis, broccoli, spinach, water melon, squash, cabbage, tomato, potato, yam, capsicum, tobacco, cotton, okra, apple, rose, strawberry, alfalfa, bean, soybean, field (fava) bean, pea, lentil, peanut, chickpea, apricots, pears, peach, grape vine or citrus species. In one embodiment, the plant is oilseed rape.

Also included are biofuel and bioenergy crops such as rape/canola, linseed, lupin and willow, poplar, poplar hybrids, Miscanthus or gymnosperms, such as loblolly pine. Also included are crops for silage (maize), grazing or fodder (grasses, clover, sanfoin, alfalfa), fibres (e.g. cotton, flax), building materials (e.g. pine, oak), pulping (e.g. poplar), feeder stocks for the chemical industry (e.g. high erucic acid oil seed rape, linseed) and for amenity purposes (e.g. turf grasses for golf courses), ornamentals for public and private gardens (e.g. snapdragon, petunia, roses, geranium, Nicotiana sp.) and plants and cut flowers for the home (African violets, Begonias, chrysanthemums, geraniums, Coleus spider plants, Dracaena, rubber plant).

A monocot plant may, for example, be selected from the families Arecaceae, Amaryllidaceae or Poaceae. For example, the plant may be a cereal crop, such as wheat, rice, barley, maize, oat sorghum, rye, onion, leek, millet, yam, buckwheat, turf grass, Italian rye grass, sugarcane or Festuca species.

Preferably, the plant is a crop plant. By crop plant is meant any plant which is grown on a commercial scale for human or animal consumption or use.

Preferred plants are maize, wheat, rice, oilseed rape, sorghum, soybean, potato, tomato, grape, barley, pea, bean, field bean, lettuce, cotton, sugar cane, sugar beet, broccoli or other vegetable brassicas or poplar.

However, the invention also applies to other eukaryotic organisms. Therefore, in other embodiments, the organism is a yeast or fungus. For example, many of processes mediated in the brewing, baking and fermenting industries are very dependent on temperature responses of the cultured yeast strains. Since the H2A.Z nucleosome eviction pathway is extremely highly conserved across the eukaryotes, this provides a means to alter the responses of commercially important yeasts to temperature in a targeted fashion using the methods described herein.

The different ways in which the method for altering temperature sensing and the method for altering thermosensory response are envisaged are set out below.

According to the methods of the invention, the presence of H2A.Z in the nucleosome can be modified in a number of ways. These include altering, decreasing or inhibiting expression of genes that encode H2A.Z. Various methods known to a skilled person can be used to achieve this, for example using RNA interference (siRNA) technology. Alternatively, the presence of H2A.Z in the nucleosome can be modified by altering the status of H2A.Z, for example the post-translational modification status of H2A.Z. This includes post-translational modifications such as acetylation, ubiquitination and methylation. The presence of H2A.Z in the nucleosome can also be modified by altering assembly of H2A.Z into the nucleosome or eviction of H2A.Z from the nucleosome.

Thus, in one embodiment of the methods of the invention, the invention relates to modifying the presence of H2A.Z in the nucleosome by decreasing the level of H2A.Z peptide or protein in said organism said method comprising decreasing or inhibiting the expression of one or more nucleic acids encoding for H2A.Z. According to this embodiment, the level or amount of H2A.Z peptide or protein is reduced, for example so that substantially no H2A.Z peptide or protein is produced in said organism. Thus, no or little H2A.Z can be incorporated into the nucleosome. The reduced level or substantial lack of H2A.Z in the nucleosome influences temperature sensing and transcriptional control of genes that respond to changes in temperature and therefore temperature dependent developmental processes. This leads to the induction of a constitutive warm temperature response even in cold temperature conditions.

As a skilled person will appreciate, different methods known in the art can be employed to decrease or inhibit the expression of one or more nucleic acids encoding for H2A.Z according to the methods of the invention. For example, said nucleic acid(s) may be silenced using RNA interference. This comprises for example expression of a simple antisense RNA, hairpin RNA, a natural or artificial microRNA or other RNA interference methods known in the art. Plants with reduced or no expression of H2A.Z encoding nucleic acid(s) may also be selected from a mutagenised population through Targeting-induced Local Lesions IN Genomes (TILLING) (Till et al., 2006).

H2A.Z is universally conserved in eukaryotic organisms and has been identified in mammals, birds, Drosophila, yeast, tetrahymenea and plants.

In plants, there are several nucleic acids that encode H2A.Z. For example, in Arabidopsis, the four H2A.Z genes are HTA8 (At2g38810), HTA9 (At1g52740), HTA4 (At4g13570) and HTA11 (At3g54560).

H2A.Z has been identified in different plants and nucleic acid sequences encoding H2A.Z can be accessed on public databases. Plant H2A.Zs are very well conserved across different taxa, including algae, maize, mosses, rice and sorghum. H2A.Z sequences from algae, mosses, rice, sorghum have conserved N-terminal acetylatable lysines (K) as in Arabidopsis. A comparison of the peptide sequences encoding H2A.Z in Arabidopsis, rice and maize shows that these sequences are highly conserved (see FIG. 11). Thus, a skilled person would be able to modify any plant H2A.Z according to the methods disclosed herein.

In another embodiment of the methods of the invention, post-translational modification of H2A.Z is modified to alter the presence of H2A.Z in the nucleosome and/or alter thermosensory responses in the organism. This can for example be achieved as set out below.

For example, the extent to which H2A.Z is acetylated can be modified. Acetylation reversibly modifies histones and thus chromatin structure. Acetylated chromatin is less tightly folded and therefore more accessible to interacting proteins. Site specific acetylation leads to activation of transcription whereas deacetylation represses transcription. Hyper-acetylated histone loosens contact with DNA, thus making nucleosome core particles less rigid.

Histone tails are normally positively charged due to amine groups present on their lysine (K) and arginine (R) amino acid residues. These positive charges help the histone tails to interact with and bind to the negatively charged phosphate groups on the DNA backbone. Acetylation of lysine (K), which occurs normally in a cell, neutralizes the positive charges on the histone by changing amines into amides and decreases the ability of the histones to bind to DNA. This decreased binding allows chromatin expansion, permitting transcription to take place.

The acetylation of histones is regulated by histone acetyltransferase enzymes (HAT). HATs are enzymes that acetylate conserved lysine amino acids on histone proteins by transferring an acetyl group from acetyl CoA to form ε-N-acetyl lysine. Acetylation can be reversed by histone deacetylation (HDAC) enzymes. HDACs remove acetyl groups, increasing the positive charge of histone tails and encouraging high-affinity binding between the histones and DNA backbone. The increased DNA binding condenses DNA structure, preventing transcription. Acetylation occurs predominantly in the highly conserved N-terminus of histones and at lysine (K) residues. H2A.Z sequence is highly conserved amongst eukaryotic organisms and comprises a number of acetylation sites. Arabidopsis H2A.Z proteins contain at least 4 to 5 potentially acetylatable K residues in its N-terminal tail, a feature that is conserved across different H2A.Z sequences across different plant species. Thus, according to the methods of the invention, the conserved acetylatable K residues in the N-terminal tail of plant H2A.Z proteins can be targeted.

According to the methods of the invention, one or more acetylation sites of H2A.Z can be targeted to decrease or increase the degree of acetylation or to substantially prevent acetylation of H2A.Z thereby altering the presence of H2A.Z in the nucleosome and/or altering thermosensory responses in the organism.

For example, site-directed mutagenesis of a gene sequence encoding for a H2A.Z peptide or protein can be used to change one or all of the K residues to a non-acetylatable residue (H2A.Z^{N}). For example, the non-acetylatable residue may be arginine (R) or another amino acid residue.

Mutagenesis of acetylatable K residues reduces or prevent acetylation. Reduced acetylation sites prevent the access of HATs, subsequent acetylation, transformation of chromatin into a less rigid structure and transcription of genes that are transcribed at elevated temperature conditions. Thus, reducing acetylation sites produces a H2A.Z^{N} that has a high affinity for DNA, and will not be evicted from the nucleosome at higher temperatures, preventing the transcription of genes that would normally be transcribed at higher temperatures and therefore developmental responses that would normally occur in response to higher temperatures.

In another embodiment, a gene sequence encoding a H2A.Z peptide or protein can be modified to introduce further acetylation sites resulting in a hyper-acetylated H2A.Z protein. This facilitates release of H2A.Z from the nucleosome. H2A.Z absence from the nucleosome results in a constitutive warm temperature response even at colder temperature, as the transcription of genes that are responsive to higher temperature is induced whilst the transcription of genes that are responsive to cold temperature is repressed.

The altered gene sequences described above can be expressed in the organism using expression vectors commonly known in the art. The mutated sequence may be part of an expression cassette comprising a promoter driving expression of said sequence. Said promoter may be the endogenous promoter, a constitutive promoter, or a tissue specific promoter. Using a tissue specific promoter, it is possible to drive expression of the transgene in a tissue specific way thus altering temperature sensing in a particular tissue. In plants, the tissue specific promoter may for example be selected from the TaPR60 Promoter that has been demonstrated to be specifically active in endosperm transfer cells in wheat and barley and starchy endosperm in rice (Kovalchuk et al., 2009), the OsPR602 and OsPR9a promoters from rice which are specific for endosperm transfer cells (Li et al., 2008), the granule-bound starch synthase 1 (gbss1) promoter which is specific to the grain filling process in wheat (Kluth et al., 2002), the napA promoter from rape for seed specific expression in crucifers, the E4, E8 or 2a11 promoter from tomato or the AGPL1 promoter from water melon for fruit specific expression and the TA29 promoter from tobacco or the A9 promoter from Arabidopsis for anther/tapetum specific expression.

Overexpression using a constitutive promoter in plants may be carried out using a strong promoter, such as the cauliflower mosaic virus promoter (CaMV35S), the rice actin promoter, the maize ubiquitin promoter, the rice ubiquitin rubi3 promoter or any promoter that gives enhanced expression. Alternatively, enhanced or increased expression can be achieved by using transcription or translation enhancers or activators and may incorporate enhancers into the gene to further increase expression. Furthermore, an inducible expression system may be used, such as a steroid or ethanol inducible expression system in plants.

In another embodiment of the methods of the invention, enzymes that acetylate or de-acetylate H2A.Z are targeted to alter the presence of H2A.Z in the nucleosome and/or alter thermosensory responses in the organism according to the methods of the invention.

In one embodiment of the methods of the invention, acetylation of the H2A.Z peptide or protein may be decreased or inhibited by decreasing or inhibiting HAT activity. For example, one or more nucleic acid sequence that encodes a HAT enzyme may be silenced using RNA interference. A construct directed against a conserved region of the HAT family may be used to decrease the levels of HAT transcript. Also, HAT activity may be decreased or inhibited by altering a nucleic acid sequence that encodes a HAT enzyme by site directed mutagenesis to decrease activity of the enzyme. Plants with reduced or little expression of HAT encoding nucleic acid(s) and/or mutated HAT protein(s) with decreased or no enzyme activity may be selected from a mutagenised population through TILLING (Till et al., 2006).

This will reduce the amount of acetylation of H2A.Z and thus prevent eviction of H2A.Z from the nucleosome. This leads to a decrease of the expression of genes requiring H2A.Z eviction from the nucleosome for their expression and thus a decrease or inhibition of developmental responses that would normally be induced by high temperature.

In another embodiment, expression of nucleic acid sequence that encodes a HAT enzyme is increased, for example by introducing into said organism one or more nucleic acids encoding HAT or otherwise altering HAT activity, for example by mutagenesis of one or more nucleic acid sequence encoding HAT. The expression of the nucleic acid sequence may be driven by the endogenous, constitutive or a tissue specific promoter. This will increase the amount of acetylation and thus increase release of H2A.Z from the nucleosome.

In another embodiment, the acetylation status of the H2A.Z peptide or protein in the nucleosome may be increased by decreasing or inhibiting HDAC activity. Decreasing or inhibiting HDAC activity prevents or reduces the removal of the acetyl group from H2A.Z. This results in reduced binding between H2A.Z and DNA and thus release of H2A.Z from the nucleosome and thus expression of genes that respond to high temperatures.

According to another embodiment of this aspect of the invention, histone deacetylase (HDAC) enzymes are expressed at enhanced levels or HDAC activity is otherwise altered, for example by mutagenesis of one or more nucleic acids encoding HAT. This prevents release of the H2A.Z from the nucleosome from the nucleosome as acetyl groups are removed. For example, one or more genes encoding for a HDAC enzyme may be constitutively expressed in the organism. A tissue specific promoter may be used.

H2A.Z is assembled into the SWR1 nucleosome by the chromatin re-modelling machinery. The components of this complex have been shown to be highly conserved amongst eukaryotes. For example, ARP6 in Arabidopsis has been shown to be responsible for inserting H2A.Z into the nucleosome in plants and is part of the SWR-1 complex. Depletion of ARP6 in mutants results in the failure to incorporate H2A.Z into chromatin and constitutive expression of the warm temperature transcriptome.

Several plant ARP6 homologues have been identified and are highly evolutionarily conserved across the plant kingdom. For example, analysis of the respective nucleic acid sequences for ARP6 homologue of rice, maize, capsella, and mosses reveals that these are highly similar to that of Arabidopsis indicating conservation across plants. Thus, a skilled person would be able to identify and modify an ARP6 homologue according to the methods of the invention.

By modifying components of the chromatin re-modelling machinery, for example a histone chaperone, the insertion or assembly of H2A.Z into the nucleosome can be prevented. Thus, in one embodiment of the methods of the invention, the presence of H2A.Z in the nucleosome can be altered by modifying the components of the chromatin re-modelling machinery. For example, in one embodiment, expression of one or more genes that encode one or more proteins involved in the insertion of H2A.Z into the nucleosome can be inhibited, for example by RNA interference. This leads to a lack of H2A.Z in the nucleosome and thus a constitutive warm temperature response. Factors that may be targeted include histone chaperones and members of the SWR-1 complex. For example, in plants ARP6 or a homologue or orthologue thereof may be targeted. Other targets may include histone chaperones that promote assembly, such as NAP1, CHZ1 or a homologue or orthologue thereof.

In another embodiment of the methods of the invention, components of the chromatin remodelling machinery, for example a histone chaperone, may be targeted to prevent disassembly of H2A.Z from the nucleosome. For example, genes that encode such factors may be silenced.

As mentioned elsewhere, the presence or absence of H2A.Z in the nucleosome induces thermosensory responses. As stated above, in one aspect, the invention therefore relates to a method for altering thermosensory responses in a eukaryotic organism, for example a plant, said method comprising decreasing the level of H2A.Z in said organism or altering post-translational modification of H2A.Z or a combination thereof.

The ways of decreasing the level of H2A.Z in said organism or altering post-translational modification of H2A.Z are explained in detail above and apply to the different embodiments of this aspect of the invention. For example, the expression of one or more nucleic acids encoding for H2A.Z may be decreased or inhibited as explained above. For example, said nucleic acid is silenced using RNA interference.

In another embodiment, post-translational modification such as acetylation may be changed as explained above. For example, site-directed mutagenesis of a gene sequence encoding for a H2A.Z peptide or protein can be used to change one or all of the K residues to a non-acetylatable residue (H2A.Z^{N}) or to introduce further acetylation sites. Moreover, as detailed above, the expression of one or more nucleic acid sequence encoding HDACs or HATs may be decreased, inhibited or enhanced to alter thermosensory responses.

For example, a mutant H2A.Z protein may be produced by expressing a nucleic acid sequence encoding a H2A.Z protein wherein said nucleic acid sequence has been altered to remove one or more acetylation sites, for example by altering a codon encoding an acetylatable K residue in said nucleic acid sequence by replacing or altering a nucleotide within said codon to produce a protein with non acetylatable residue. In another embodiment, a mutant H2A.Z protein may be produced by expressing a nucleic acid sequence encoding for a H2A.Z protein wherein said nucleic acid sequence has been altered to introduce one or more additional acetylation sites. As explained elsewhere, promoters according to the invention may be either the endogenous, a constitutive or a tissue specific promoter.

Altering post-translational modification according to the method may also comprise inhibiting or decreasing the expression of one or more nucleic acid sequence encoding for a HDAC enzyme to reduce or inhibit deacetylation of H2A.Z or expressing one or more nucleic acid sequences encoding a HDAC enzyme to increase removal of acetyl groups in H2A.Z.

In another embodiment, altering post-translational modification according to the method may also comprise inhibiting or decreasing the expression of one or more genes encoding for a HAT enzyme to inhibit acetylation or expressing one or more nucleic acid sequences encoding a HAT enzyme to increase acetylation of H2A.Z. Expression of said nucleic acid sequences according to the method may be driven by the endogenous, a constitutive, inducible or a tissue specific promoter.

Thermosensory responses are those responses that are observed in an organism in response to changes in temperature. These include changes in gene transcription. In plants, genes such as HSP70 are up-regulated at higher temperatures. HSP70 gene transcription follows a proportionate pattern with increasing temperature over a wider range (about 12° C. to 37° C.) changes in gene transcription (of the temperature transcriptome) and can therefore be used as a marker gene which indicates up or down-regulation of the temperature transcriptome. Transcriptional changes also influence developmental processes and in plants an increase of temperature affects a number of developmental processes, such as flowering, hypocotyl elongation and petiole growth. It also leads to an increase in the rate of transition through different developmental phases. Juvenile to adult transition is therefore accelerated by higher ambient temperature. An increase in temperature leads to acceleration of flowering time, greater hypocotyl elongation, petiole growth, germination, and general acceleration of plant growth and development.

According to the methods of the invention, the thermosensory responses in a plant that can be altered by the method described above and said responses include one or more of and preferably all of the following developmental responses: seed dormancy release, germination, hypocotyl elongation, petiole growth, vernalisation, juvenile to adult transition, flowering, senescence and temperature-protective responses.

In one embodiment, the entirety or totality of the thermosensory response in a plant is modified. Thus, substantially all of the developmental and transcriptional responses that a plant shows in response to changes in temperature can be altered by increasing or decreasing the level of H2A.Z in said organism or altering post-translational modification of H2A.Z or a combination thereof.

Another aspect of the invention relates to a method for inducing a warm temperature response in a eukaryotic organism comprising inhibiting or decreasing the presence H2A.Z in the nucleosome. The response may be constitutive and is induced even in temperatures which would normally be perceived as cold temperatures by said plant. The method may include modifying the expression of one or more gene encoding for H2A.Z, increasing the acetylation status of H2A.Z or preventing assembly of H2A.Z into the nucleosome as described herein.

As shown herein, inhibiting or decreasing the presence H2A.Z in the nucleosome in plants leads to an induction of responses that would normally be seen under high temperature conditions (about ≧22° C.). The mutant plants phenocopy wild type plants growing at a higher temperature (around 5° C. higher than the actual temperature). Although a skilled person would appreciate that the temperature that determines a warm temperature response varies depending on the plant species, generally speaking, a warm temperature in plants response is a response that is shown at about 20° C. to about 24° C., for example about ≧22° C. in Arabidopsis.

Thus, in one embodiment, crops that require a certain threshold temperature for proper growth and yield can be manipulated using the methods of the invention to reduce that temperature threshold. This is of particular benefit for crops that are grown in the greenhouse.

Another aspect of the invention relates to a method for inducing a cold temperature response in a eukaryotic organism comprising inhibiting or decreasing the eviction of H2A.Z from the nucleosome. The response may be constitutive. The method may include preventing acetylation status of H2A.Z or preventing disassembly of H2A.Z from the nucleosome as described herein. For example in plants, the mutant plants phenocopy wild type plants growing at a lower temperature (≦17° C.). Although a skilled person would appreciate that the temperature that determines a cold temperature response varies depending on the plant species, generally speaking, a cold temperature response in plants is therefore a response that is shown at about 15° C. to about 19° C., for example about ≦17° C. in Arabidopsis. Other plants have different temperature optima as a skilled person will appreciate.

The invention also relates to methods for producing a eukaryotic organism, for example a plant, that shows a constitutive warm temperature response, even in cold temperatures and under conditions in which such response would not normally be displayed. Thus, in one embodiment, the invention relates to a method for producing a plant that shows a constitutive warm temperature response said method comprising inhibiting or decreasing the presence H2A.Z in the nucleosome. The method may include modifying the expression of one or more genes encoding for H2A.Z, increasing the acetylation status of H2A.Z or preventing assembly of H2A.Z into the nucleosome as described herein.

In another aspect, the invention relates to a method for producing a eukaryotic organism, for example in a plant, that shows a constitutive cold temperature response, even in high temperatures and under conditions in which such response would not normally be displayed. Thus, in one embodiment, the invention relates to a method for producing a plant that shows a constitutive cold temperature response said method comprising inhibiting or decreasing the eviction of H2A.Z from the nucleosome. The method may include preventing acetylation status of H2A.Z or preventing disassembly of H2A.Z from the nucleosome as described herein.

The invention also provides a plant H2A.Z molecule in which some or all of the acetylatable lysines of the molecule have been replaced by non-acetylatable amino acid residues.

The invention further provides a plant comprising a novel H2A.Z molecule in which some or all of the lysines of the molecule have been replaced by non-acetylatable amino acid residues or a nucleic acid sequence encoding such a molecule.

Furthermore, the invention provides a plant comprising a non-acetylatable version of histone H2A.Z, an over-expressed histone deacetylase enzyme sufficient to remove histone acetylation marks in H2A.Z, or a down regulated histone acetyltransferase activity.

According to the aspects above, the plant may be selected from the plants recited elsewhere herein.

Another object of this invention is to provide a method to enhance yield in crop plants by altering temperature sensing as according to the embodiments described above. In one embodiment, this aspect relates to a method to enhance grain-fill, grain composition or grain quantity in grain crops or to prevent premature flowering or bolting. In particular, the method relates to enhancing yield in crop plants by altering temperature sensing to allow said crop to reach its yield potential even at temperatures higher than those at which grain-fill is typically limited, or to avoid premature bolting/flowering following exposure to temperature conditions that typically cause such premature bolting.

As mentioned above, many harmful eukaryotic micro-organisms use temperature perception as a signal to induce pathogenesis, for example Histoplasma capsulatum and Candida albicans (Nguyen and Sil, 2008). Therefore, the ability to selectively perturb temperature perception in these organisms would provide means to prevent pathogenesis. Since the components of the deposition and eviction machinery are highly conserved, they make very good targets for small molecule inhibition. Furthermore, since the yeast would still be viable without undergoing pathogenesis, selection for resistant strains would be weak.

Therefore, in another aspect, the invention also relates to a method for inhibiting pathogenesis of a eukaryotic micro-organism comprising altering temperature sensing in a eukaryotic organism comprising modifying the presence of H2A.Z in the nucleosome. Ways of altering temperature sensing are described herein. Moreover, small molecule therapies can be used to target the system.

Other objects and advantages of this invention will be appreciated from a review of the complete disclosure provided herein and the appended claims.

While the present invention has been generally described above, the following non limiting examples are provided to further describe the present invention, its best mode and to assist in enabling those skilled in the art to practice this invention to its full scope. The specifics of these examples should not be treated as limiting, however.

### EXAMPLES

### Experimental Procedures

Unless otherwise stated herein, materials and methods used were as described here:

### Genetic Screens for Temperature Perception

All experiments were carried out in Col-0 background. A forward genetic screen of fast neutron mutagenized population of HSP70::LUC reporter strain was carried out to identify mutants perturbed in the ambient temperature perception pathway (seeds irradiated at HAS KFKI-Atomic Energy Research Institute, Hungary). Seven-day-old seedlings were incubated at 12° C. for 48 h before shifting to 27° C. for three hours and screening for LUC expression by spraying with Luciferin. The entr mutations were identified as having significantly higher LUC induction than the reporter line. ENTR1 was identified as ARP6 through a microarray-based transcript analysis. Genomic fragment of containing ARP6 genomic fragment (P_{ARP6}::ARP6) was used to complement entr1 mutants.

### Transcript Analysis

Transcript analyses were done on total RNA was extracted using Trizol (Invitrogen) Labeled targets were hybridized to Affymetrix ATH1 array according to the manufacturer's instructions. Microarray data were analyzed using GenespringGX 7.3 (Agilent).

### Chromatin Immuno-Purification (ChIP)

ChIP was performed as described (Gendrel et al., 2002) with minor modifications. Seven-day-old seedlings grown at 17° C. were shifted to 27° C. for 2 hours and ChIP experiments were performed in parallel on chromatin from both temperatures. For H2A.Z and H3 ChIP, cross-linked chromatin was fragmented with 0.2 units of Micrococcal nuclease (Sigma) in 1 ml of MNase digestion buffer (10 mM Tris-HCl [pH 8.0], 50 mM NaCl, 1 mM β-mercaptoethanol, 0.1% NP40, 1 mM CaCl₂ and 1× protease inhibitor cocktail [Roche]). Digestion was stopped using 5 mM EDTA. HTA11, one of the Arabidopsis H2A.Z homologues, was GFP tagged and ChIP was performed using GFP polyclonal antibody (Abcam, ab290). Histone H3 dynamics were assayed using H3 antibody (Abcam, ab1791). For RNA polymerase II occupancy dynamic experiments chromatin was fragmented by sonication in lysis buffer (10 mM Tris-HCl [pH 8], 150 mM NaCl, 1 mM EDTA [pH 8], 0.1% deoxycholate and 1× protease inhibitor cocktail) and ChIP was performed using monoclonal antibody to RNA polymerase II CTD repeat YSPTSPS (Abcam, ab817). All ChIP experiments were performed in buffer containing 10 mM Tris-HCl [pH 8.0], 5 mM EDTA [pH 8.0], 150 mM NaCl, 1% Triton X-100 and 1× protease inhibitor cocktail. Relative enrichment of associated DNA fragments were analysed by quantitative PCR (qPCR). All oligonucleotide sequences used for target DNA detection and quantification in ChIP experiments are given in supplementary material. Each ChIP experiment was repeated at least three times and the data presented are from a representative experiment.

### Nucleosome Positioning

Micrococcal nuclease (MNase) digestion of enriched chromatin followed by qPCR using tiled oligonucleotides surrounding the transcription start site (TSS) was used for nucleosome positioning and analysis. Oligos were designed to have 95-110 bp amplicons every 35-45 bp in the HSP70 promoter. For nucleosome positioning, chromatin from seven-day-old seedlings of Col-0 and entr1 grown at varying temperatures (17° C., 22° C., 27° C.) were digested with MNase and mononucleosome sized fragments were gel purified and used in qPCR. Relative nucleosome occupancy was represented as fraction of uncut chromatin DNA and was plotted against the HSP70 gene position with respect to the TSS for each primer pair where the position denotes the center of each amplicon.

### Nucleosome Purification by Hydroxyapatite Chromatography

Seedlings growing at 17° C. were frozen in liquid nitrogen without cross-linking and chromatin was prepared as for the ChIP experiments. Following MNase digest for 15 min at 17° C., chromatin fragments were bound to hydroxyapatite and nucleosomes devoid of linker histones and other associated proteins were purified according to (Brand et al., 2008). Purified nucleosomes were buffer exchanged to 10 mM Tris-HCl (pH 8.0), 1 mm EDTA (pH 8.0), 25 mM NaCl and 1× protease inhibitor cocktail. Approximately 500 ng equivalent of nucleosomal DNA was assayed for restriction enzyme accessibility at 17° C. and 27° C. The fraction of the input nucleosomal DNA protected was obtained using qPCR for HSP70-1 and +1 nucleosomes. Data represented are normalized against the +1 nucleosome of At4g07700 that does not have a restriction site.

### Example 1

### HSP70 is an Output of the Ambient Temperature Sensing Pathway

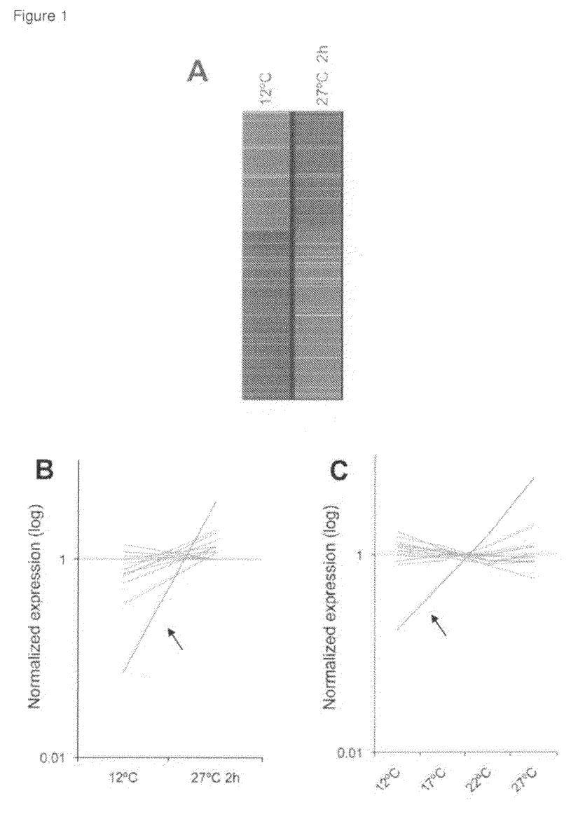

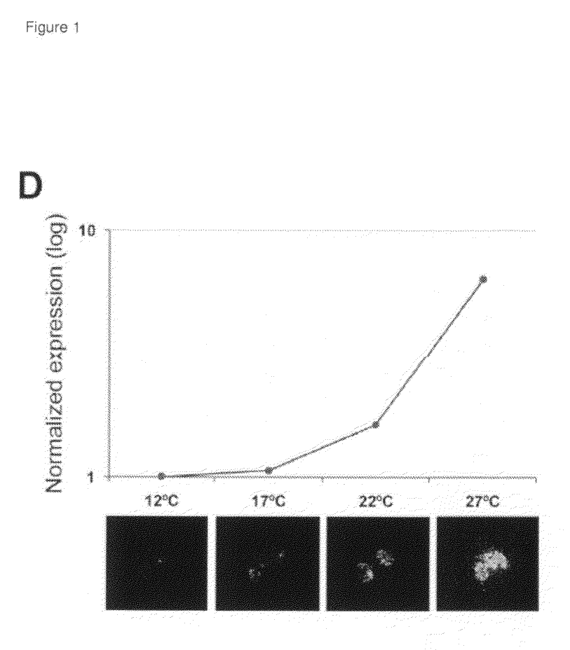

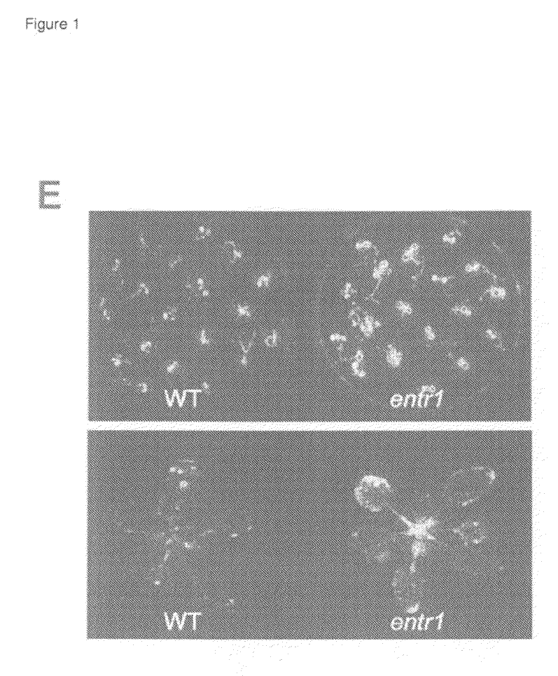

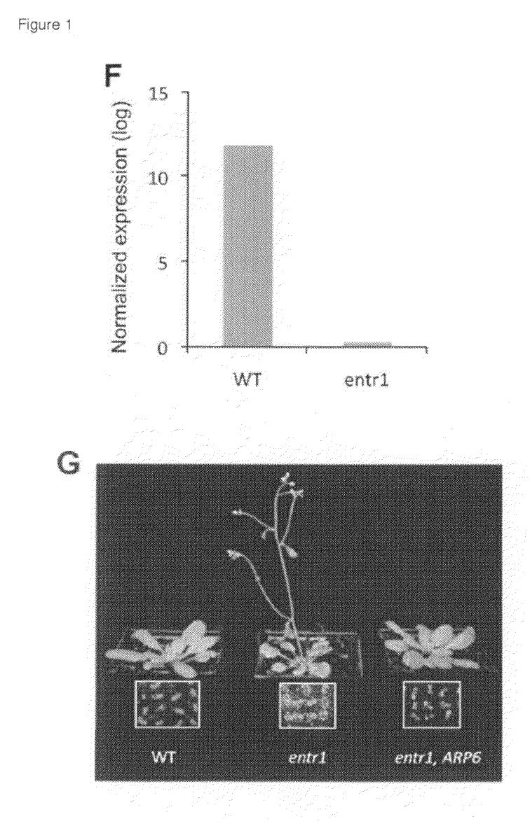

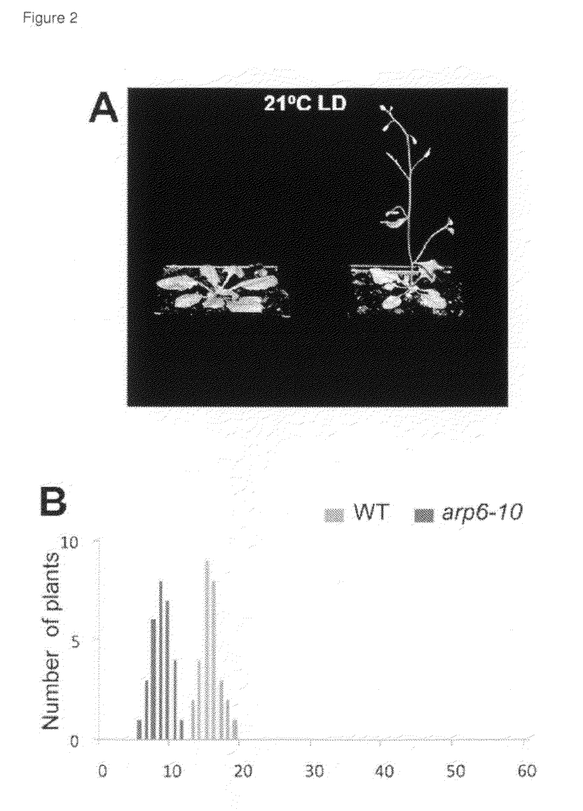

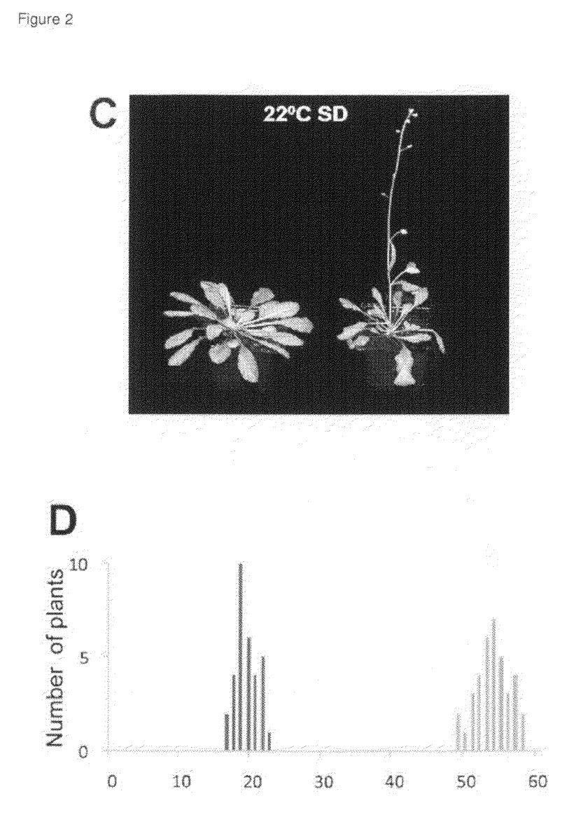

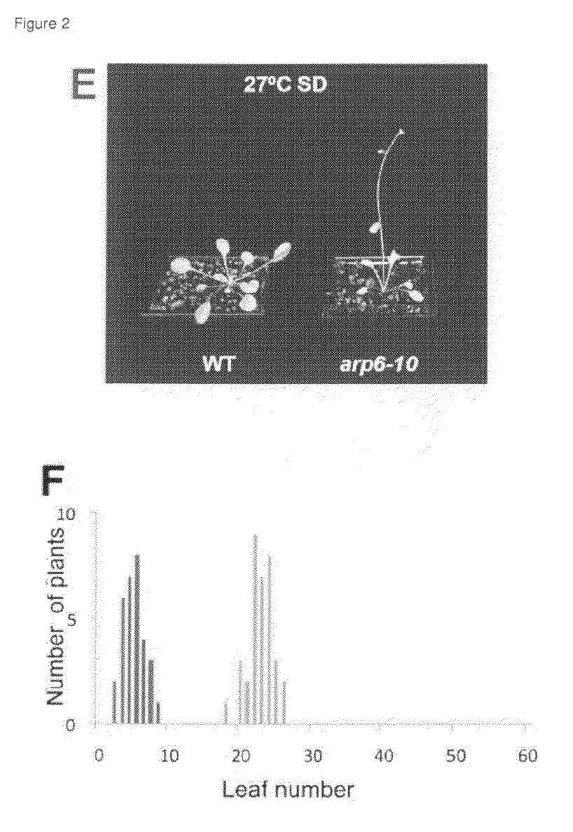

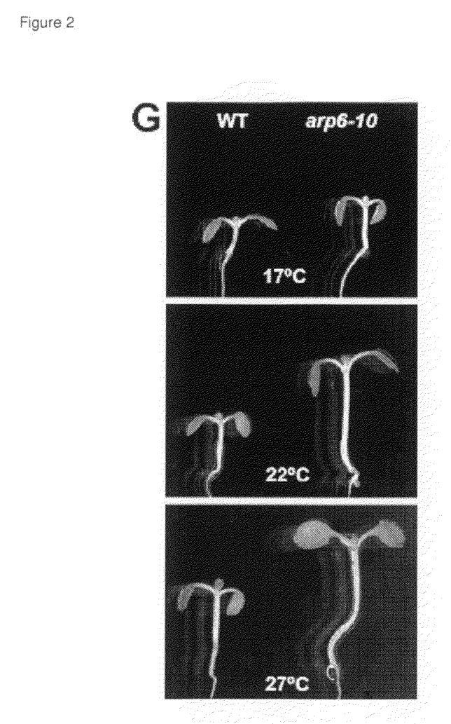

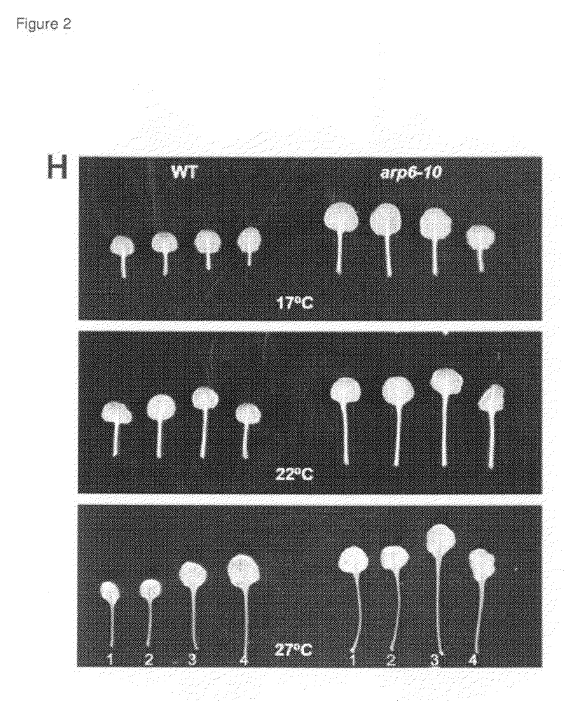

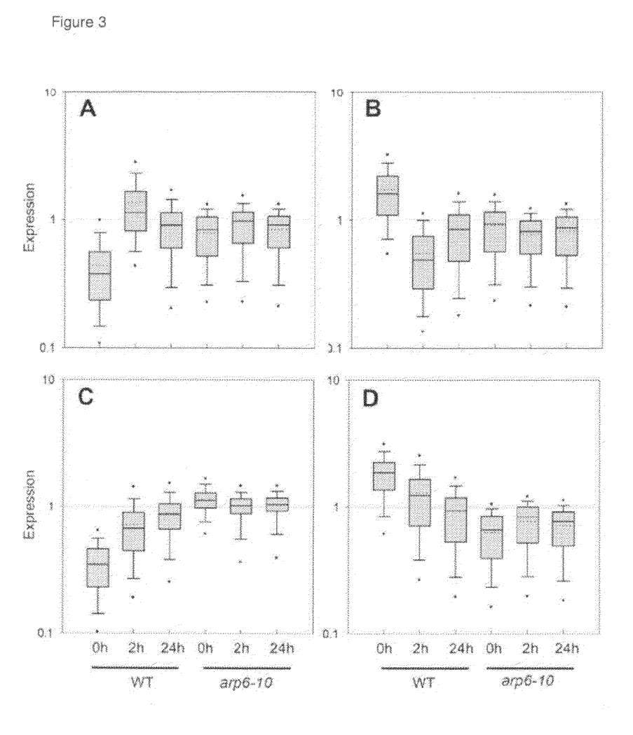

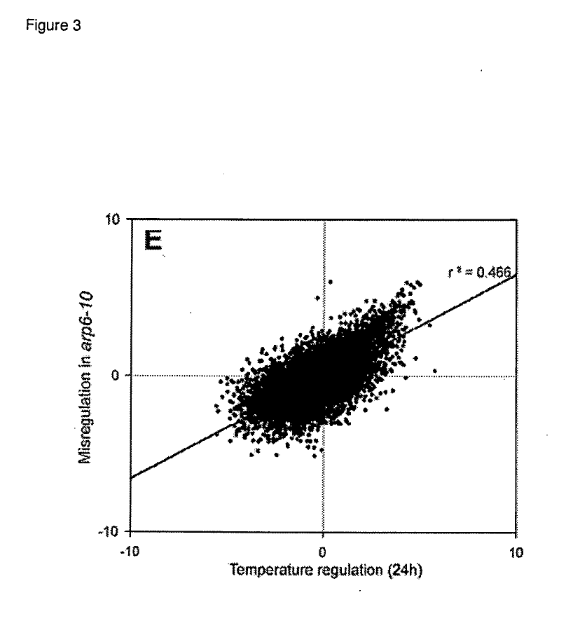

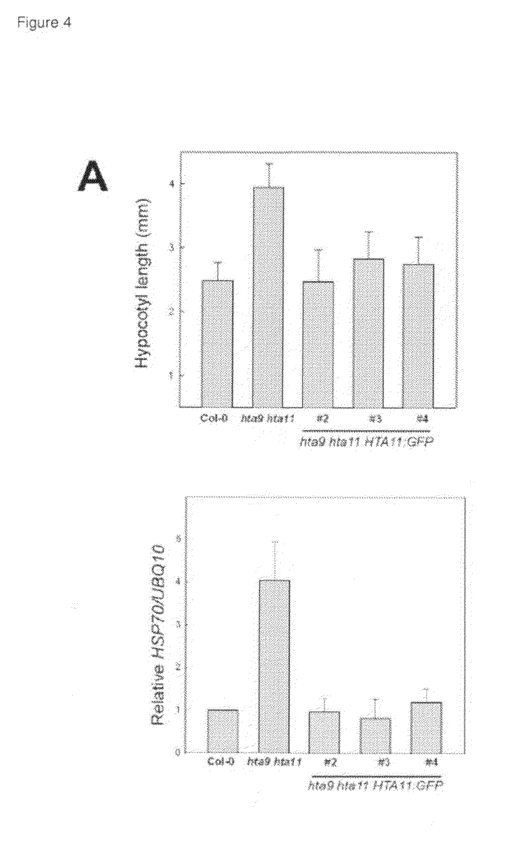

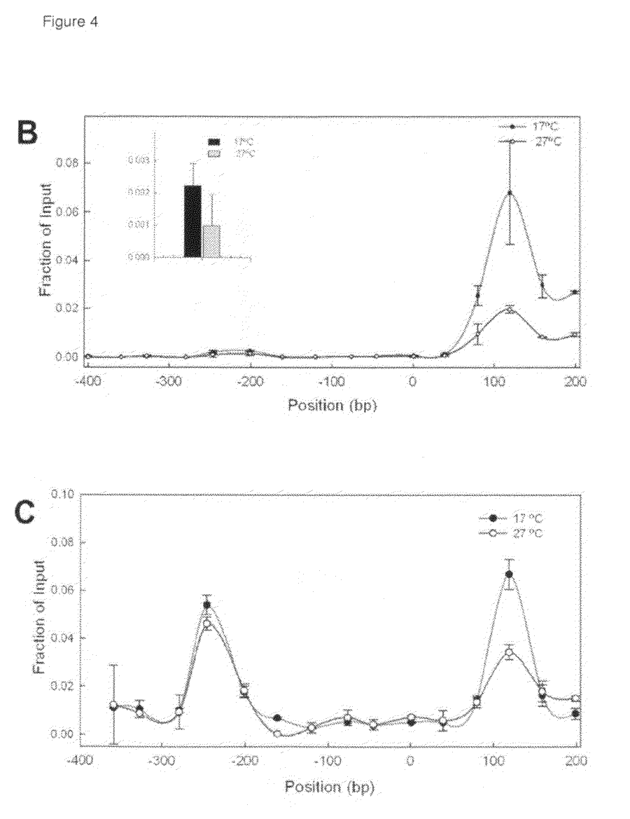

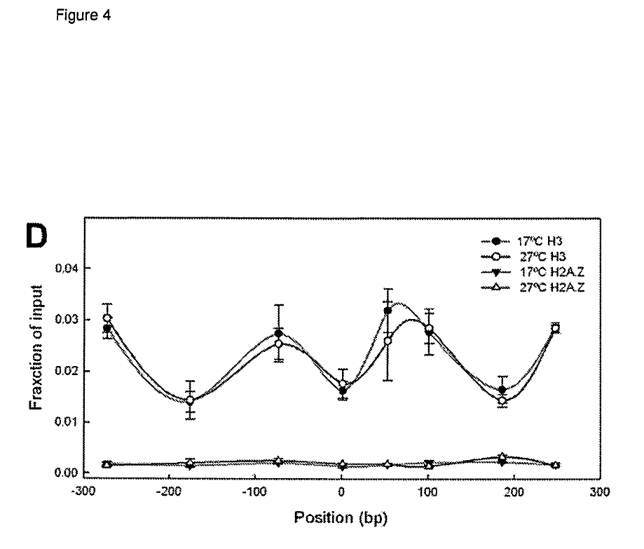

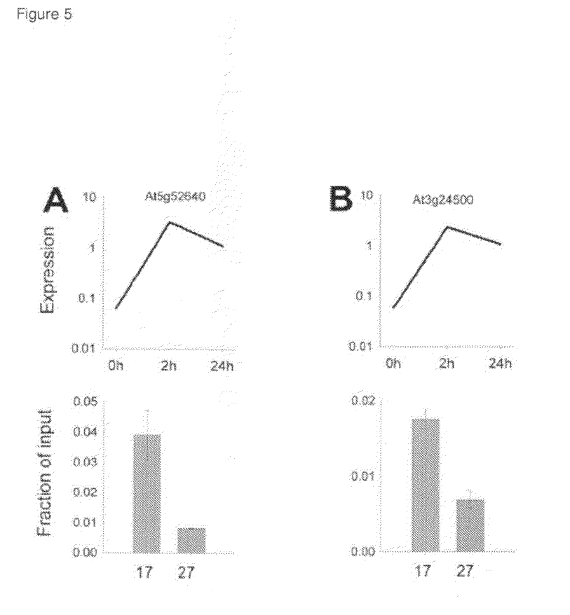

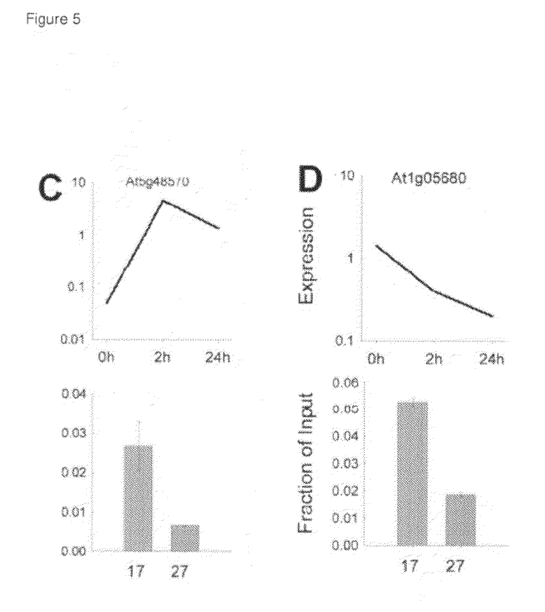

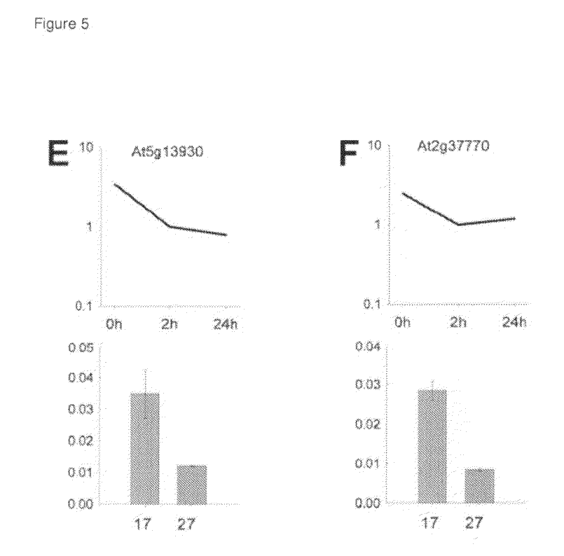

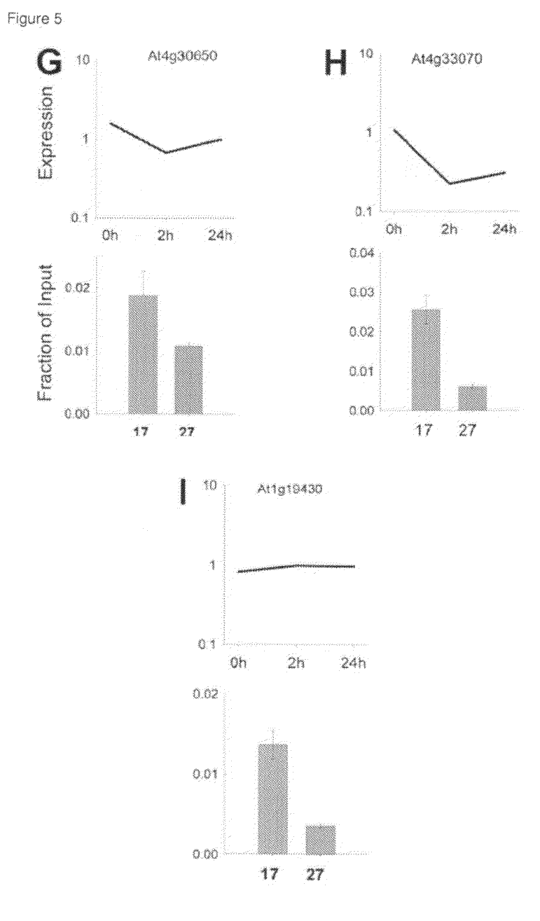

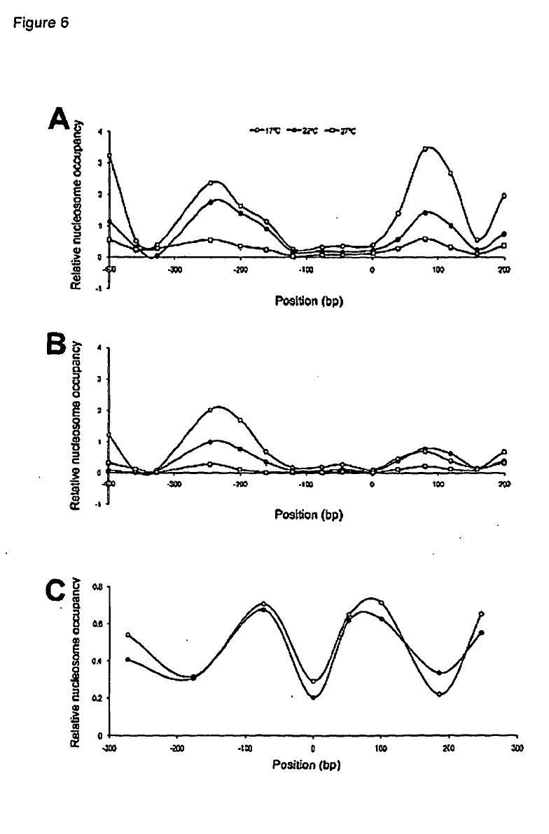

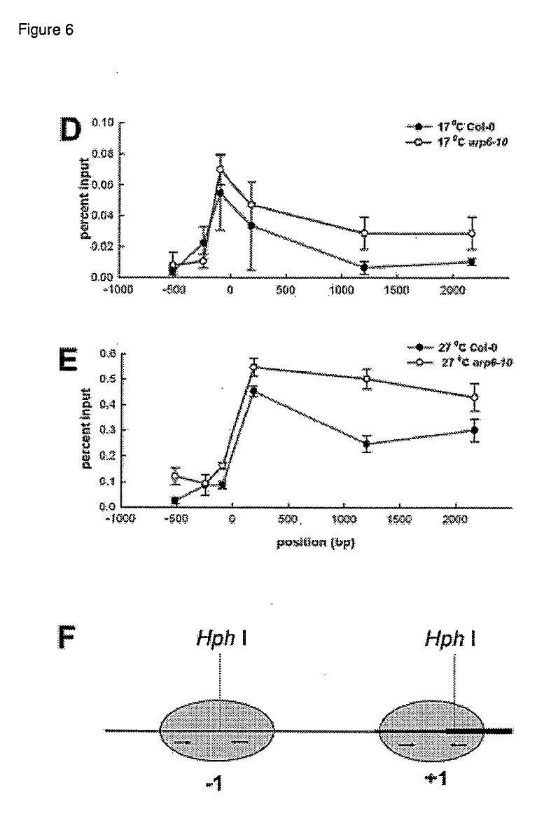

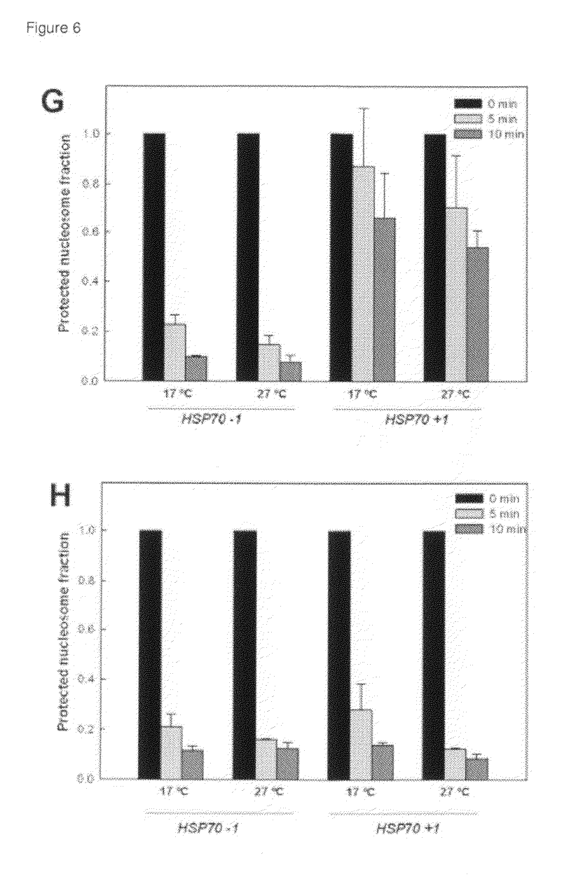

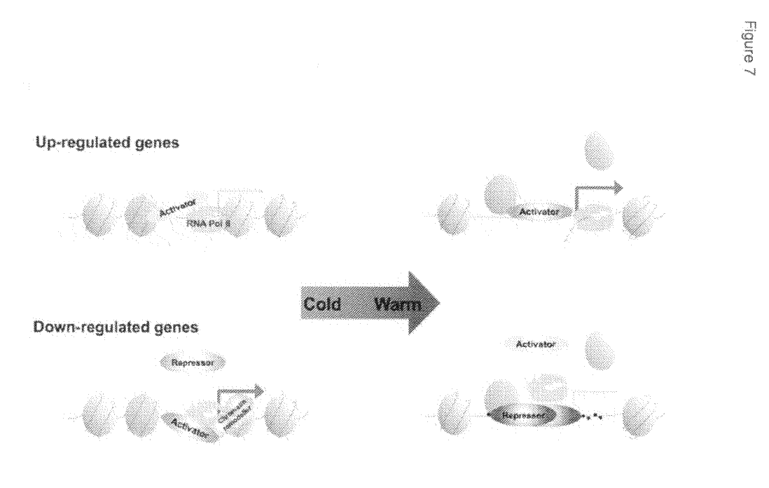

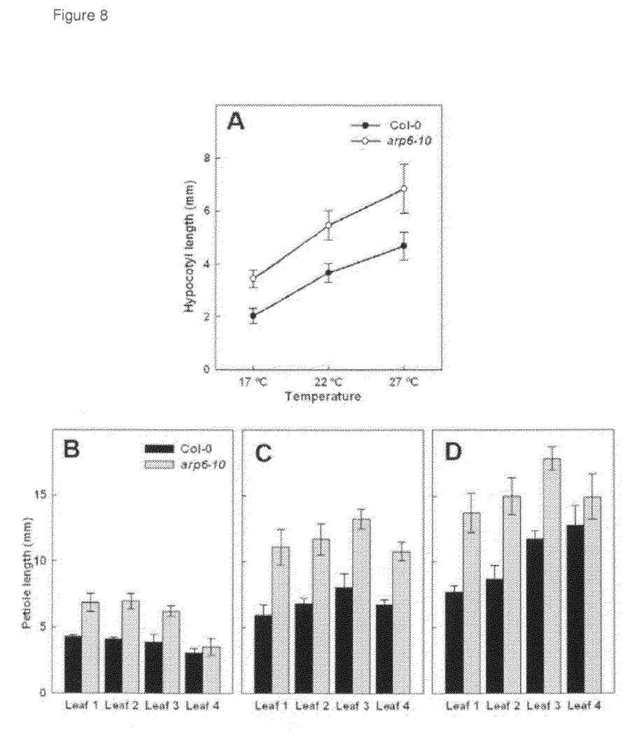

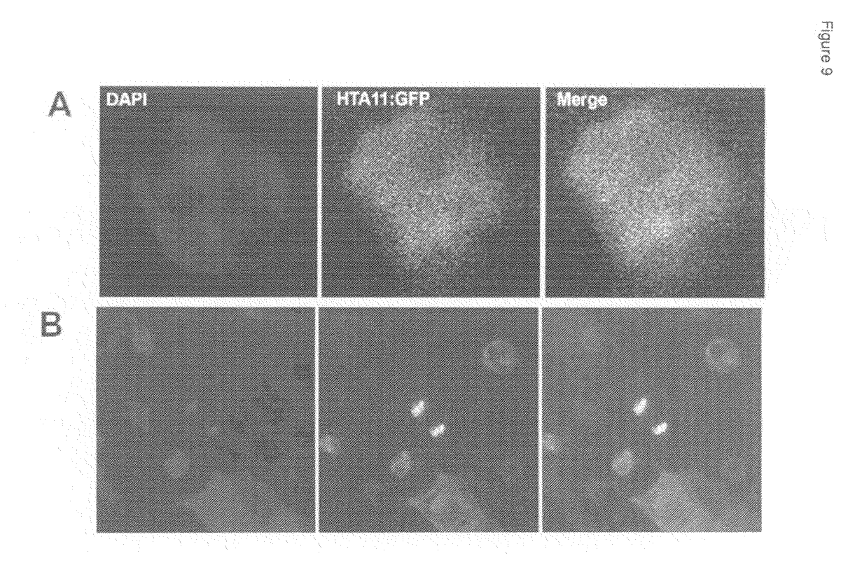

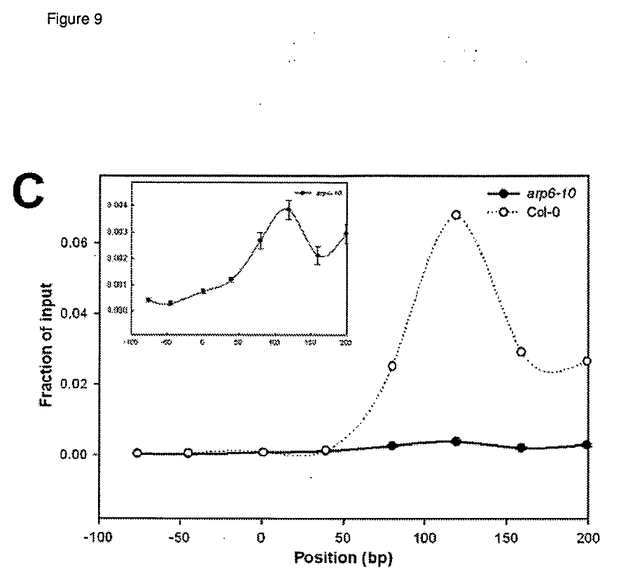

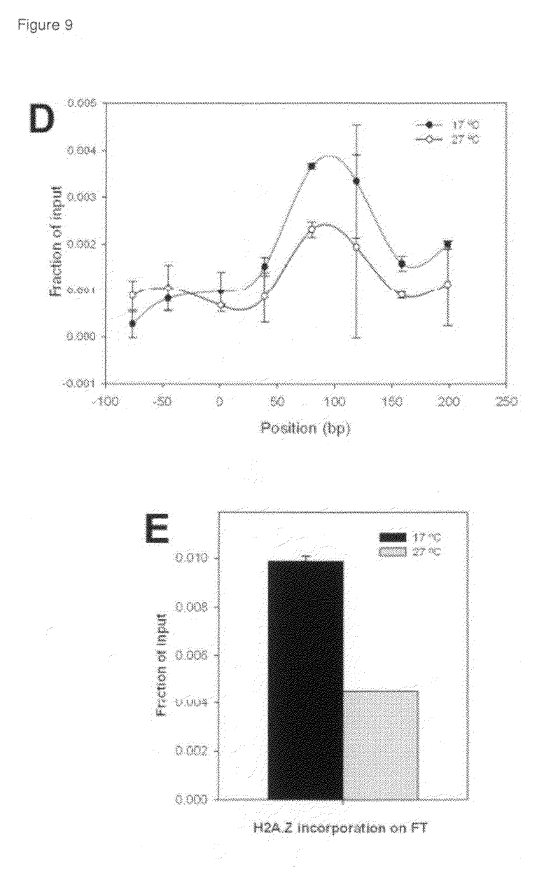

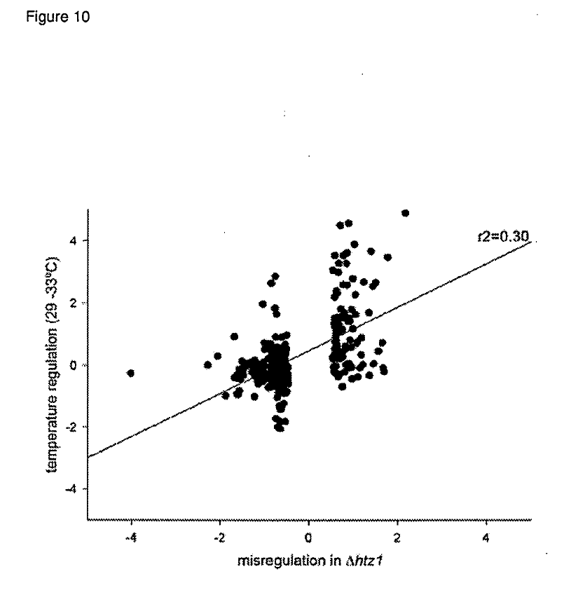

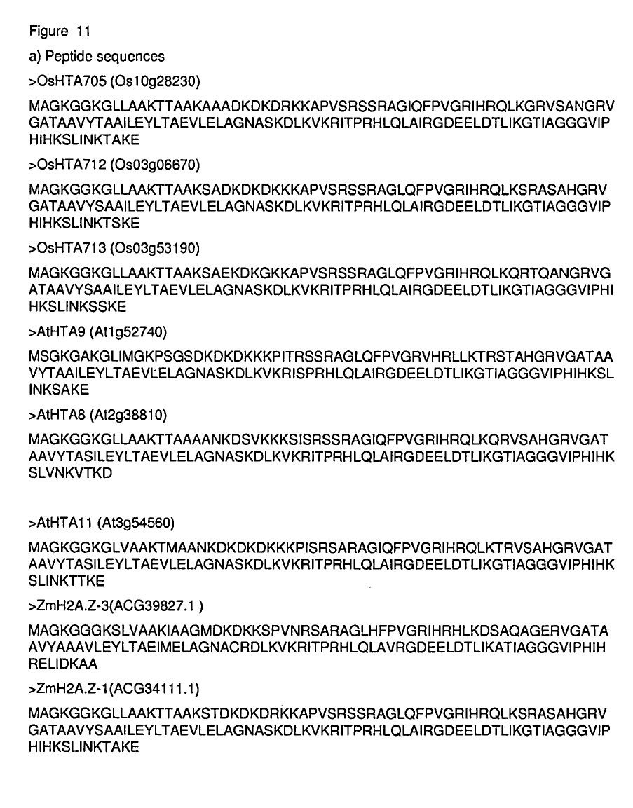

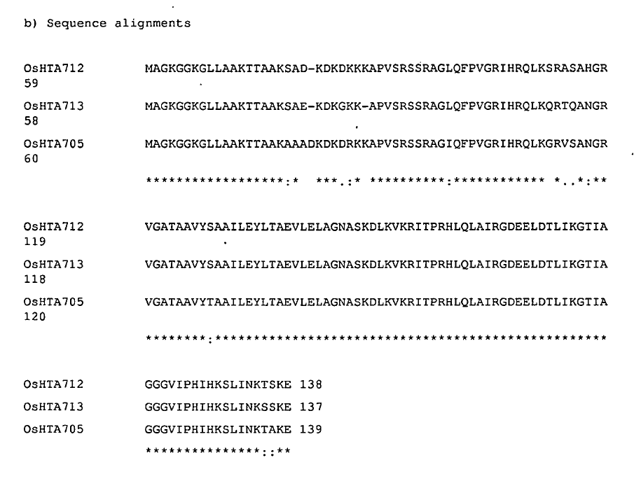

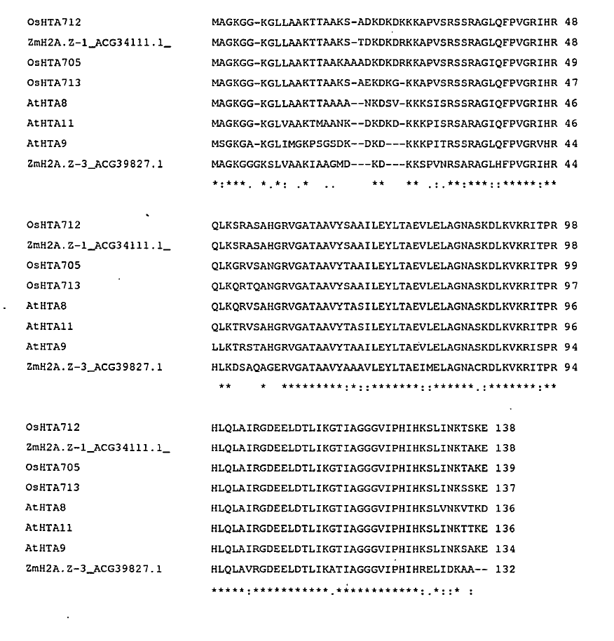

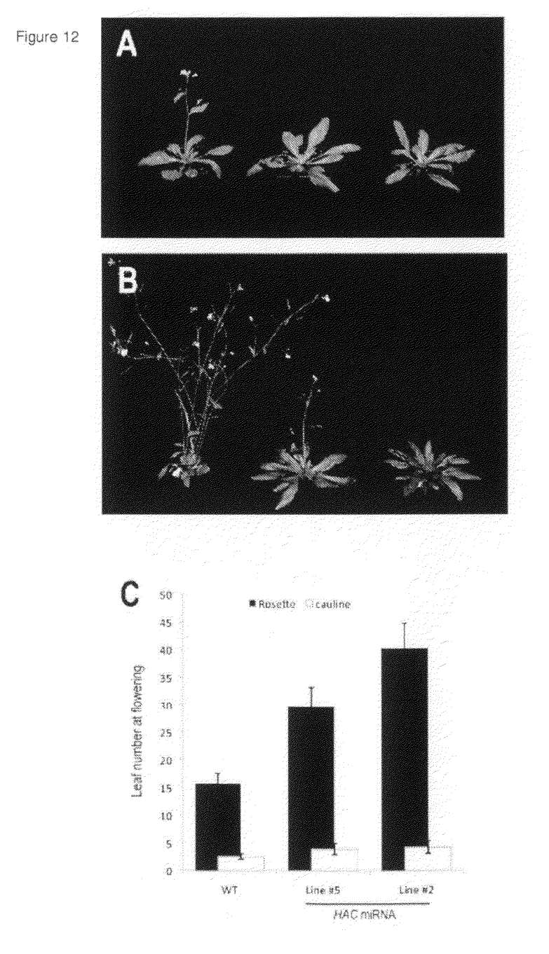

To identify the major ambient temperature responses in seedlings, we analysed the transcriptomes of 12 day old plants grown at 12° C. and shifted to 27° C. We found that 2454 genes are upregulated at least 2-fold under these conditions and 2880 genes are 2-fold down-regulated (the ambient temperature transcriptome; FIG. 1A). As in similar studies, there was not a significant induction of stress markers, suggesting that this temperature range is not causing heat stress (Balasubramanian et al., 2006). We found that HSP70 (At3g12580), is strongly up-regulated at higher temperature (FIG. 1B). While this transcriptome is characterised by a response to ambient temperature change, we wanted to determine if any of these genes were also responsive to differences in constant growth temperature, since the transcriptional output of a thermosensory pathway should be different at various constant temperatures. We therefore analysed the behaviour of the induced ambient temperature transcriptome genes in publicly available data for plants grown at 12° C., 17° C., 22° C. and 27° C. (NASCARRAYS-147 (http://affymetrix.arabidopsis.info/narrays/experimentpage.pl?experimentid=147).). We found HSP70 is expressed at a level proportionate to the ambient temperature within this temperature range (FIG. 10). While these experiments were performed on seedlings, HSP70 also shows these expression dynamics in the adult plant (Balasubramanian et al., 2006). We therefore used a fusion of the HSP70 promoter to Luciferase (HSP70::LUC) to monitor the activity of the endogenous gene non-destructively. HSP70::LUC recapitulates the expression of HSP70 (FIG. 1D), providing us with a dynamic and sensitive assay for temperature perception status in planta, independent of high temperature stress (Larkindale and Vierling, 2008; Sung et al., 2001).

### Example 2

### ARP6 is in the Ambient Temperature Sensing Pathway that Controls Flowering

To identify genes required to control the ambient temperature transcriptome, we screened 2,600 individual M2 families of fast neutron irradiated HSP70::LUC seedlings. Two mutations, entr1 and entr2 (enhanced temperature response1 and 2), displayed a constitutively higher LUC expression (FIG. 1E). Genetic analysis using a complementation cross revealed that these mutations are allelic. Transcript based cloning revealed that the ARP6 transcript is absent in both entr1 and entr2 (FIG. 1F). Transformation of entr1 with a genomic fragment of ARP6 is able to rescue both the HSP70::LUC expression level as well as the altered development of entr1 (FIG. 1G), confirming that it is a new arp6 allele. We will refer to entr1 and entr2 as arp6-10 and arp6-11 respectively. Arabidopsis mutants in ARP6 have been identified in flowering time screens (Choi et al., 2005; Deal et al., 2005; Martin-Trillo et al., 2006). To determine if ARP6 acts in the ambient temperature pathway for flowering, we analysed flowering in arp6-10. Consistent with earlier reports, arp6-10 flowers earlier at 21° C. in long and 22° C. in short days (FIG. 2A-2D). We then studied the response of arp6-10 to 27° C. in short days, since the thermal induction of flowering is most pronounced in short days (Balasubramanian et al., 2006). Remarkably, arp6-10 flowers with about 5 leaves in short days at 27° C. (FIGS. 2E and 2F). ARP6 therefore acts in the ambient temperature pathway that controls flowering.

### Example 3

### ARP6 Controls Developmental Responses to Ambient Temperature Globally

Specific adaptive changes occur to plant architecture in response to higher ambient temperature, including increases in hypocotyl growth and petiole elongation (Gray et al., 1998; Koini et al., 2009). We analysed these traits to see if arp6-exhibits a global high temperature response in its architecture as well as HSP70 expression and flowering time. We find that architecture responses are strongly enhanced in the arp6 background (FIGS. 2G and 2H and FIG. 8), such that arp6 plants grown at 17° C. exhibit greater hypocotyl elongation and petiole growth than wild-type plants grown at 22° C., with an equivalent difference between 22° C. and 27° C. These phenotypes have been shown to be dependent on the PIF4 transcription factor (Koini et al., 2009), so it is not surprising that we still observe a temperature-induced difference, even in the arp6-10 mutant. A functional ARP6 is required however for controlling the correct level of expression of these phenotypes. A longstanding observation in plant biology is that thermal time is a key measure for the rate of transition through the developmental phases, and that phase transition is accelerated by higher ambient temperature (Poethig, 2003). In addition to the flowering time acceleration in arp6-10, we observe a more rapid juvenile to adult transition (data not shown), consistent with earlier studies (Martin-Trillo et al., 2006). Thus arp6-10 causes a specific up-regulation of all the developmental decisions known to be regulated by temperature that we have examined. Plants deficient in ARP6 therefore display a constitutive warm temperature developmental program.

### Example 4

### The Ambient Temperature Transcriptome is Globally Misregulated in arp6

To determine if the global mis-regulation of developmental responses in the absence of ARP6 occurs at the transcriptional level, we compared the ambient temperature transcriptomes of arp6-10 and wild-type. While 2,454 genes are upregulated 2-fold or more on shifting from 12° C. to 27° C. in wild-type (FIG. 3A, grey bars), we observe this ambient temperature transcriptome is consistently expressed in arp6-10, even at 12° C. (FIG. 3A, blue bars). Interestingly, this effect reflects more than a constitutive up-regulation of gene expression per se, since the 2880 genes that are down regulated at least 2-fold on increasing temperature in wild-type (FIG. 3B, grey bars) are also constitutively repressed in arp6-10. Thus, transcriptionally, the arp6-10 mutant at 12° C. resembles a plant at a higher temperature. In a reciprocal analysis, transcripts that are 2-fold more highly expressed at 12° C. in arp6-10 compared to wild-type (FIG. 3C, blue bars) are strongly induced by higher ambient temperature in wild-type, while those genes that are repressed in arp6-10 compared to wild-type at 12° C. (FIG. 3D, blue bars), are transcriptionally repressed by higher temperature in wild-type (FIG. 3D, grey bars). These results show that in the absence of ARP6, the ambient temperature regulated genes are constitutively mis-expressed. To determine what proportion of the entire ambient temperature transcriptome is ARP6 dependent we compared the change of gene expression in response to higher ambient temperature in wild-type with the change in expression resulting from arp6-10 at constant temperature. Remarkably, there is a strong correlation for the entire transcriptome: a functional ARP6 allele accounts for almost half of the ambient temperature transcript responses (R²=0.466, P<0.001). ARP6 is therefore necessary for coordinating the transcriptome in response to ambient temperature.

### Example 5

### H2A.Z Occupancy Changes with Temperature

ARP6 encodes a subunit of the SWR1 complex that is conserved among eukaryotes and is necessary for inserting the alternative histone H2A.Z into nucleosomes in place of H2A (Deal et al., 2007; Kobor et al., 2004; Krogan et al., 2003; Mizuguchi et al., 2004). There are several H2A.Z family members in Arabidopsis, and we find the hta9 hta11 double mutant phenocopies arp6-10, showing the early flowering and architectural responses as well as increased HSP70 expression. This indicates that the temperature responses of arp6-10 result from a failure to incorporate H2A.Z-containing nucleosomes into the genome. Global analyses in yeast, Drosophila, C. elegans, Arabidopsis and mammalian cells have revealed that H2A.Z-containing nucleosomes are especially enriched at the +1 position near the transcriptional start site (Creyghton et al., 2008; Mavrich et al., 2008; Raisner et al., 2005; Whittle et al., 2008; Zhang et al., 2005; Zilberman et al., 2008). H2A.Z has been implicated in maintaining promoters of quiescent genes in a poised state ready for appropriate transcription (Li et al., 2005). A possible explanation for the central role of ARP6 in regulation of the temperature transcriptome is that H2A.Z-containing nucleosomes confer a temperature dependence on transcription. To test if H2A.Z dynamics change in response to temperature, we used chromatin immunopurification (ChIP) to follow H2A.Z occupancy at the HSP70 promoter in response to different ambient temperatures. We used HTA11:GFP expressed under the endogenous HTA11 promoter for our ChIP experiments. This construct complements the hta9 hta11 double mutant (FIG. 4A), showing that HTA11:GFP is functional. Microscopy of HTA11:GFP reveals incorporation into euchromatin as in other studies (FIGS. 9A and 9B) (Zilberman et al., 2008).

ChIP analysis revealed a greatly depleted H2A.Z occupancy at the HSP70 promoter in arp6-10 (FIG. 9C), suggesting that the increased HSP70 expression in arp6-10 is a consequence of this change in chromatin architecture, as indicated by the hta9 hta11 phenotype. To see if temperature affects H2A.Z occupancy at HSP70, we compared seedlings grown at 17° C. with seedlings shifted to 27° C. for two hours. Consistently, at 17° C., where expression of HSP70 is low, we observe a high occupancy of H2A.Z at the +1 nucleosome (FIG. 4B). This occupancy is however very temperature dependent, with H2A.Z occupancy decreasing sharply at 27° C. compared to 17° C. (FIG. 4B). We have validated our ChIP results using an H2A.Z antibody (Deal et al., 2007) that gives similar results (FIG. 9D). These results are consistent with a model where H2A.Z occupancy is rate limiting for the transcription of temperature responsive genes, and the occupancy of H2A.Z responds to temperature. To determine whether the behaviour we observe is specific to H2A.Z or is a general property of nucleosomes in response to temperature, we carried out parallel ChIP studies using histone H3 specific antibody. Upon shifting to 27° C., H3 levels at the +1 nucleosome drop, though not to the extent of H2A.Z (FIG. 4C). Interestingly at the −1 nucleosome, where H2A.Z occupancy is reduced, H3 levels did not drop significantly. This suggests that H2A.Z-containing nucleosomes exhibit specific dynamic responses to temperature. Since RNA Pol II must negotiate +1 nucleosomes for transcription to occur, this suggests a model where gene transcription may be controlled by the temperature responsive occupancy of +1 nucleosomes.

The changes we observe in H3 occupancy at HSP70 may be a consequence of H2A.Z nucleosomes having a temperature specific response. We therefore extended our analysis to the TSS of another locus, At4g07700, chosen since it does not contain H2A.Z (Zilberman et al., 2008) and it is largely transcriptionally silent, avoiding complicating factors arising from RNA Pol II mediated displacement of nucleosomes, as well as being predicted to have a well ordered nucleosome structure using a recent model (Kaplan et al., 2009). ChIP analysis with H3 specific antibody showed the predicted nucleosome occupancy. ChIP analysis revealed there was no significant change in H3 occupancy at this locus upon shifting to 27° C. (FIG. 4D). This is consistent with our findings for the −1 nucleosome of HSP70 where there is little or no H2A.Z incorporation, and H3 levels do not show significant temperature responsive dynamics. These results suggest that H2A.Z is the major determinant temperature responsiveness of chromatin.

To test if this response is unique to HSP70, we examined the TSS proximal regions corresponding to the presumptive +1 nucleosomes of a number of other temperature up-regulated genes (FIG. 5A-5C). Consistently, all these genes show a sharp reduction in H2A.Z occupancy at 27° C. Since it has been shown that FT is an essential output of the thermosensory pathway for mediating early flowering, we sought to see if H2A.Z is present in the FT promoter and if it is thermoresponsive. Although FT is not significantly expressed in seedlings, eviction of H2A.Z may be necessary to confer transcriptional competence on the FT locus. Analysis of global H2A.Z occupancy data (Zilberman et al., 2008) revealed that H2A.Z is enriched in the FT promoter. We therefore assayed for H2A.Z changes at FT in response to temperature change. Consistently, we observe H2A.Z depletion from the FT promoter at higher temperatures (FIG. 9E), suggesting an explanation for the acceleration of flowering in arp6.

### Example 6

### Eviction of H2A.Z Occurs Independently of Transcription

We have shown that H2A.Z occupancy decreases dynamically with increasing temperature at the +1 nucleosomes of temperature-induced genes. Since temperature-dependent histone eviction may be a consequence of greater transcriptional activity, and is not necessarily the cause, we sought to determine if H2A.Z eviction occurs independently of transcription. To test this, we analysed genes that are enriched for H2A.Z in their promoters and are either down regulated or show constant expression in response to increased temperature. We then performed ChIP to assay H2A.Z occupancy in response to ambient temperature change (FIG. 5D-5I). We observed a significant decrease in H2A.Z occupancy across all the genes examined, independent of their transcriptional response to temperature. This result suggests H2A.Z-containing nucleosomes have a binding affinity or occupancy as measured by ChIP that varies with temperature. This is independent of transcription, which is analogous to the behaviour of nucleosomes at the Hsp70 locus of Drosophila (Petesch and Lis, 2008). Since the presence of H2A.Z-containing nucleosomes is rate-limiting for the expression of the majority of the genes in the ambient temperature transcriptome, this suggests that H2A.Z responses to temperature play a key role in integrating temperature information.

### Example 7

### The HSP70 +1 Nucleosome Shows a Constitutive Temperature Response in arp6

Since we have found that H2A.Z nucleosomes are evicted at higher temperature, we analysed the nucleosome structure of the HSP70 promoter at different temperatures by studying MNase accessibility. MNase digests nucleosome-free DNA and the linker regions between nucleosomes, while the DNA of the nucleosomes themselves is protected from digestion (Petesch and Lis, 2008). For plants grown at 17° C., 22° C. and 27° C., we observed a sharp drop in nucleosome occupancy with greater temperature (FIG. 6A), which helps us understand how HSP70 transcription increases with temperature. By comparison, the +1 nucleosome of HSP70 in the arp6 background is constitutively in the open conformation, even at 17° C. (FIG. 6B), at which temperature there is significant nucleosome occupancy in wild-type. At higher temperatures, we also observe a decrease in the −1 occupancy, which is likely a consequence of greater transcriptional activity. This decrease in −1 occupancy appears to be H2A.Z independent. Consistently, the nucleosome structure of At4g07700, which does not contain H2A.Z nucleosomes, does not change with temperature (FIG. 6C).

The results so far are consistent with a model where expression of HSP70 may be regulated at the level of nucleosome occupancy. For RNA Pol II to transcribe target genes, it must be able to overcome the physical barrier posed by nucleosomes (Knezetic and Luse, 1986; Lorch et al., 1987).

Numerous studies indicate that nucleosomes at genes induced to undergo transcription dissociate or partially unwrap from their DNA, allowing passage of RNA Pol II, and the presence of nucleosomes, especially the +1 nucleosome near the transcriptional start site, can be rate limiting for controlling transcription (Boeger et al., 2008; Boeger et al., 2003). Under heat shock conditions, nucleosomes at the Drosophila Hsp70 locus are lost from the body of the gene in advance of the arrival of RNA Pol II, indicating that the nucleosomes are able to play a role in regulating transcription (Petesch and Lis, 2008).

RNA Pol II ChIP was used to study transcriptional dynamics at HSP70 in wild-type and arp6-10. At 17° C. in wild-type, we observed a greater proportion of RNA Pol II present at the TSS compared to in the body of the gene. By comparison, in arp6 a greater proportion of RNA Pol II is present in the body of the gene (FIG. 6D). At 27° C. there is a strong shift of RNA Pol II occupancy, with the highest peak now occurring downstream of the TSS. Since this new RNA Pol II peak corresponds to the region occupied by the +1 nucleosome, it is likely that in wild-type at non-inductive conditions the +1 nucleosome plays a role in maintaining transcription in a poised state. Upon increasing temperature, the presence of Pol II on the gene body increases considerably (FIG. 6E). This is consistent with studies in C. elegans and Drosophila, which have suggested that H2A.Z+1 nucleosomes may maintain RNA Pol II in a poised state (Mavrich et al., 2008; Whittle et al., 2008). The presence of H2A.Z containing nucleosomes is therefore rate-limiting for the upregulation of HSP70.

### Example 8

### H2A.Z Nucleosomes Wrap DNA More Tightly

We have found that in the absence of H2A.Z incorporation, canonical H2A containing nucleosomes are unable to prevent constitutive expression of the warm temperature transcriptome of both up- and down-regulated genes. This difference could result directly from a difference in the behaviour of H2A.Z nucleosomes, or may reflect the effect of other factors recruited by H2A.Z. To distinguish between these two possibilities, we purified nucleosomes from plants grown at 17° C. using hydroxyapatite chromatography following a partial MNase digest. It has recently been demonstrated that RNA Pol II does not actively unwrap nucleosomes, but depends on fluctuations that locally unwrap DNA for it to transcribe its template (Hodges et al., 2009). Since the invasion of RNA Pol II into nucleosomal DNA is the rate limiting step for transcription, we assayed the exposure of restriction enzyme sites occluded by H2A.Z and H2A nucleosomes at 17° C. and 27° C. (FIGS. 6F and 6G). This assay provides a direct measure of the degree of local unwrapping and exposure of nucleosomal DNA (Polach and Widom, 1995). The H2A-containing −1 nucleosome of HSP70 displays a greater accessibility as compared to the H2A.Z containing +1 nucleosome at both 17° C. and 27° C. This greater accessibility of H2A-containing nucleosomes provides an explanation for the greater transcription of HSP70 in the arp6 background where H2A.Z is not incorporated into chromatin. The rate of exposure of these sites appears to increase with higher temperature, suggesting that local fluctuations of the nucleosomal DNA increase with temperature. Since there might be subtle differences in the accessibility of the Hph I site between the −1 and +1 nucleosomes, we purified nucleosomes from arp6. In this material both nucleosomes contain H2A, and as we would predict, they are both equally accessible (FIG. 6H). These results show that H2A.Z containing nucleosomes wrap DNA more tightly, and higher temperatures may overcome this. Since these nucleosomes were purified from plant material, we do not exclude that post-translational modifications, for example acetylation, may be involved in modulating this response. Importantly, our assay is performed on highly purified material, indicating that the dynamic responses we observe are an inherent property resulting from the nucleosome-DNA interactions. Our finding that the degree of local unwrapping of DNA on H2A.Z containing nucleosomes is reduced compared to H2A nucleosomes suggests a direct mechanism by which transcription can be adjusted according to the temperature of the cell.

### Example 9

### The Role of H2A.Z in Controlling the Temperature Transcriptome is Conserved

Since temperature changes represent a fundamental challenge to all sessile organisms, we sought to determine if control of temperature transcriptomes might be a conserved function of the H2A.Z nucleosomes in eukaryotes generally. To test this, we analysed the role of the budding yeast H2A.Z, Htz1, in temperature signalling. The Htz1 transcriptome has been previously determined (Meneghini et al., 2003), and we therefore tested to see if those genes that are mis-regulated in the htz1Δ background also show a corresponding change when wild-type yeast are shifted from 29° C. to 33° C. (Gasch et al., 2000). Strikingly, we see a significant correlation (R²=0.30, P<0.001), with genes that are up-regulated at higher temperatures tending to also be upregulated in htz1Δ and vice versa (FIG. 10). This is the same relationship we have observed in Arabidopsis (FIG. 3E), showing that the loss of H2A.Z containing nucleosomes phenocopies cells at higher temperature. These data suggest that warm temperature signals are mediated through H2A.Z nucleosomes in yeast as well as plants.

### Example 10

### Expression Non-Acetylatable (Mutant) Version of H2A.Z

Arabidopsis encodes three major H2A.Z genes (HTA8, HTA9 and HTA11). Mutation of the two major genes HTA9 and HTA11 (in hta9 hta11 double mutants) phenocopy arp6 mutant indicative of enhanced temperature response. We have been able to complement the hta9 hta11 double mutant phenotype by expressing HTA11 from its native promoter.

A 2786 bp genomic region of HTA11 (At3g54560) including 1589 bp promoter region and coding sequences (genomic coordinates Chromosome 3: 20194677-20197463) was amplified using oligos W1160 (5′CACCGAAATGTTTTTCTCTACG 3′) and W1161 (5′CTCCTTGGTGGTTTTGTTGA 3′). The amplified fragment was cloned into pENTR vector (invitrogen) according to the manufacturer's instructions. pENTR-HTA11 was then used for further mutagenesis experiments. PCR mediated site-directed mutagenesis was used to mutate conserved lysine residues in the N-terminal tail of HTA11.

Lysine residues at positions 4, 7, 13, 19 and 21 of HTA11 N-terminus were selected for mutagenesis. The wild type HTA11 with all five lysine residues intact is hereafter mentioned HTA11-KKKKK.

Oligos W1443(5′ TCAAGAGACATGGCAGGCAGAGGTGGAAGAGGACTCGTAGCTGCG 3′) and W1444 (5′ CGCAGCTACGAGTCCTCTTCCACCTCTGCCTGCCATGTCTCTTGA 3′) were used for targeted mutagenesis of lysine's residues 4 (K₄) and K₇ to arginines (R) using HTA11-KKKKK as template. The resulting plasmid was named HTA11-RRKKK.

Oligos W1445(5′ GGACTCGTAGCTGCGAGGACGATGGCTGCTAAC 3′) and W1446 (5′ GTTAGCAGCCATCGTCCTCGCAGCTACGAGTCC 3′) were used for targeted mutagenesis of lysine's residues 13 (K₁₃) to arginines (R) using HTA11-KKKKK as template. The resulting plasmid was named HTA11-KKRKK.

Oligos W1447(5′ GACGATGGCTGCTAACAGGGACAGAGACAAGGACAAGAAG 3′) and W1448 (5′ CTTCTTGTCCTTGTCTCTGTCCCTGTTAGCAGCCATCGTC 3′) were used for targeted mutagenesis of lysine's residues 19 and 21 (K₁₉, K₂₁) to arginines (R) using HTA11-KKKKK as template. The resulting plasmid was named HTA11-KKKRR.

Oligos W1445(5′ GGACTCGTAGCTGCGAGGACGATGGCTGCTAAC 3′) and W1446 (5′ GTTAGCAGCCATCGTCCTCGCAGCTACGAGTCC 3′) were used for targeted mutagenesis of lysine's residues 13 (K₁₃) to arginines (R) using HTA11-RRKKK as template. The resulting plasmid was named HTA11-RRRKK.

Oligos W1447(5′ GACGATGGCTGCTAACAGGGACAGAGACAAGGACAAGAAG 3′) and W1448 (5′ CTTCTTGTCCTTGTCTCTGTCCCTGTTAGCAGCCATCGTC 3′) were used for targeted mutagenesis of lysine's residues 19 and 21 (K₁₉, K₂₁) to arginines (R) using HTA11-RRRKK as template. The resulting plasmid was named HTA11-RRRRR.

Wild type (HTA11-KKKKK) and mutagenized versions of HTA11 (HTA11-RRKKK, HTA11-KKRKK, HTA11-KKKRR, HTA11-RRRKK and HTA11-RRRRR) were transferred to a binary gateway vector pW1357 by recombination so as to have c-terminus fusions of the proteins with 3×FLAG tag. FLAG tagged wild type and mutant versions of HTA11 are used to transform hta9 hta11 double mutant as well as Col-0 wild type.

### Example 11

### Knocking Out (or Knocking Down) Acetyltransferase Enzyme Gene

The HAC family of Arabidopsis histone acetyl transferase family consists of five genes At1g79000 HAC1, At1g67220 (HAC2), At1g55970 (HAC4), At3g12980 (HAC5) and At1g16710 (HAC12). Artificial microRMA (miRNA) design using Web MicroRNA Designer (http://wmd3.weigelworld.org/cgi-bin/webapp.cgi). miRNA was amplified as suggested (http://wmd3.weigelworld.org/downloads/Cloning_of_artificial_microRNAs.pdf) using oligos. Amplified miRNA was cloned into pENTR vector and there after into binary transformation vector pW545 through recombination to create 35S::HACmiRNA(pW1009). Agrobacterium strains harboring the binary vector pW1009 was used to transform Arabidopsis Col-0. Preliminary analysis of the transgenic lines revealed a delayed flowering phenotype consistent with the expectation. These transgenic lines are analysed in detail to characterize ambient temperature response.

### Example 12

### Overexpressing Deacetylase Enzymes

Protein coding sequences of one or more of the histone deacetylase (HDAC) genes are amplified from a cDNA library, and are cloned into pENTR vector and there after transferred to a binary vector under the control of a constitutive CaMV35S promoter. Arabidopsis plants were transformed with the binary vector harbouring Agrobacterium to obtain transgenic lines.

### Nucleosomes as Temperature Responsive Regulators of Transcription

Nucleosomes play an active role in controlling transcriptional processes through modulating the ability of transcription factors to access their cis elements or occluding the passage of RNA Pol II (Lam et al., 2008; Segal and Widom, 2009). Access of proteins to DNA wrapped around histones is mediated through local unwrapping events. This unwrapping is not affected by changes in ambient temperature in our study and others for H2A-containing nucleosomes (Polach and Widom, 1995). By contrast, H2A.Z-containing nucleosomes exhibit a much tighter wrapping of their DNA, consistent with studies of nucleosomal stability (Thambirajah et al., 2006). This is supported by the observation that H2A.Z-containing nucleosomes are more tightly positioned in the genome and are less fuzzy. Work on reconstituted nucleosomal arrays has also shown that the intranucleosomal interactions are stronger in H2A.Z-containing nucleosomes, while these have weaker internucleosomal interactions (Fan et al., 2002). In addition to H2A.Z-containing nucleosomes having more tightly wrapped DNA, our results suggest that the degree of unwrapping may also be responsive to temperature. This result suggests a direct mechanism by which temperature may influence gene expression, since it has been shown that RNA Pol II does not actively invade nucleosomes, but waits for local unwrapping of DNA from nucleosomes before extending transcription (Hodges et al., 2009). In this way, genes with a paused RNA Pol II will show increased transcription with greater temperature as local unwrapping is increased (FIG. 7). For those genes whose transcription decreases with temperature, we propose greater temperature increases the frequency with which transcriptional repressors may bind. The presence of H2A.Z-containing nucleosomes in these genes may prevent binding of a transcriptional repressor, or may antagonise DNA methylation which has been shown to be a role of H2A.Z in plants (Zilberman et al., 2008). Consistently, H2A.Z-containing nucleosomes have been shown to play an important role in anti-silencing (Meneghini et al., 2003).

A large number of genes that are both up- and down-regulated in arp6-10 and exhibit a corresponding change with increased temperature. Furthermore, we observe the same de-regulation of the warm temperature transcriptome in the htz1Δ mutant in yeast, suggesting this is a conserved function of H2A.Z containing nucleosomes. Importantly, there are many physiological responses to temperature, and undoubtedly many mechanisms for sensing and responding to temperature in different organisms. For example the PIF4 transcription factor mediates the high temperature architecture phenotypes in plants (Koini et al., 2009). Our mechanism for H2A.Z-containing nucleosomes provides an attractive model for how these transcriptional responses may be integrated with temperature status. On theoretical grounds it has been shown that regulating transcription at multiple levels greatly improves robustness and adaptivity (Tsankov et al., 2006).

### Connections Between Thermosensing and Flowering Pathways

Arabidopsis thaliana has a highly plastic life history, and a genetically informed photothermal model is able to explain most of the variation in flowering time for plants grown at different seasons (Wilczek et al., 2009). Acceleration of flowering in response to higher temperature requires genes in the autonomous pathway (Blazquez et al., 2003), and is dependent on increasing expression of FLOWERING LOCUS T (FT) (Balasubramanian et al., 2006; Cerdan and Chory, 2003; Halliday et al., 2003; Lee et al., 2007).

Flowering is one of the major developmental pathways regulated by ambient temperature. It has been shown that two autonomous flowering pathway genes, FVE and FCA, are necessary for mediating the ambient temperature response for flowering (Blazquez et al., 2003). Interestingly, an arp6 allele, esd1-2, is epistatic to fve and fca (Blazquez et al., 2003; Martin-Trillo et al., 2006). FVE is a homolog of the mammalian retinoblastoma-associated protein, a component of a histone deacetylase complex (Ausin et al., 2004; Kim et al., 2004). As well as affecting the autonomous pathway, which regulates flowering time through the floral pathway integrator FLOWERING LOCUS C (FLC), FVE is also involved in sensing cold temperatures (Kim et al., 2004). Taken together, these data suggest that FVE may exert its effects on temperature-dependent pathways through modulating H2A.Z, perhaps through acetylation, which affects nucleosome stability (Ishibashi et al., 2009; Thambirajah et al., 2006). Although the thermosensory flowering pathway is mediated by FT expression levels, the major regulator of FT expression in response to long photoperiods, CONSTANS (CO), is not essential for perceiving temperature, since co-1 responds to thermal induction of flowering (Balasubramanian et al., 2006). Our observation that the chromatin status at the FT locus responds to temperature and is altered in the absence of H2A.Z provides an explanation for how the thermal induction pathway may activate FT expression in response to higher temperature independently of CO. Interestingly, another temperature dependent pathway that also affects flowering time in plants, vernalisation, is also perturbed in the arp6 background (Choi et al., 2005; Deal et al., 2007). A vernalisation requirement in certain Arabidopsis genotypes is suppressed by arp6. This effect is mediated through FLC, a floral repressor whose expression is dependent on the SWR1 complex. Interestingly, in a large field experiment it was found that natural temperature fluctuations were likely to be sufficient for overcoming FLC induced repression of flowering in Arabidopsis (Wilczek et al., 2009). Temperature fluctuations of this type are likely to signal through H2A.Z dynamics, which is consistent with the observation that perturbation of H2A.Z in arp6 is sufficient to overcome a vernalisation requirement (Choi et al., 2005; Deal et al., 2005; Martin-Trillo et al., 2006).

### Temperature Perception and Climate Change

While many plants have shown a rapid acceleration of flowering time in response to climate change (Fitter and Fitter, 2002), species that do not are going locally extinct at a high rate (Willis et al., 2008). This indicates that phenological responses to temperature change are of high adaptive value. A molecular understanding of the mechanism of temperature perception will enable us to understand how different species will respond to further increases in temperature and be a key step towards breeding crops able to withstand climate change.

### Example 13

### Transformations of Barley and Brassica

Barley plants are transformed with the vector pBRACT214 for constitutive overexpression of the altered H2A.Z non-acetylatable versions under the Ubiquitiin promoter. Brassica are transformed with the vector pBRACT103 for expression of non-acetylatable H2A.Z versions of H2A.Z.

### REFERENCES

Ausin, I., Alonso-Blanco, C., Jarillo, J. A., Ruiz-Garcia, L., and Martinez-Zapater, J. M. (2004). Regulation of flowering time by FVE, a retinoblastoma-associated protein. Nat Genet 36, 162-166.
Balasubramanian, S., Sureshkumar, S., Lempe, J., and Weigel, D. (2006). Potent induction of Arabidopsis thaliana flowering by elevated growth temperature. PLoS Genet 2, e106.
Blazquez, M. A., Ahn, J. H., and Weigel, D. (2003). A thermosensory pathway controlling flowering time in Arabidopsis thaliana. Nat Genet 33, 168-171.
Boeger, H., Griesenbeck, J., and Kornberg, R. D. (2008). Nucleosome retention and the stochastic nature of promoter chromatin remodeling for transcription. Cell 133, 716-726.
Boeger, H., Griesenbeck, J., Strattan, J. S., and Kornberg, R. D. (2003). Nucleosomes unfold completely at a transcriptionally active promoter. Mol Cell 11, 1587-1598.
Brand, M., Rampalli, S., Chaturvedi, C. P., and Dilworth, F. J. (2008). Analysis of epigenetic modifications of chromatin at specific gene loci by native chromatin immunoprecipitation of nucleosomes isolated using hydroxyapatite chromatography. Nat Protoc 3, 398-409.
Cerdan, P. D., and Chory, J. (2003). Regulation of flowering time by light quality. Nature 423, 881-885.
Choi, K., Kim, S., Kim, S. Y., Kim, M., Hyun, Y., Lee, H., Choe, S., Kim, S. G., Michaels, S., and Lee, I. (2005). SUPPRESSOR OF FRIGIDA3 encodes a nuclear ACTIN-RELATED PROTEIN6 required for floral repression in Arabidopsis. Plant Cell 17, 2647-2660.
Creyghton, M. P., Markoulaki, S., Levine, S. S., Hanna, J., Lodato, M. A., Sha, K., Young, R. A., Jaenisch, R., and Boyer, L. A. (2008). H2AZ is enriched at polycomb complex target genes in ES cells and is necessary for lineage commitment. Cell 135, 649-661.
Deal, R. B., Kandasamy, M. K., McKinney, E. C., and Meagher, R. B. (2005). The nuclear actin-related protein ARP6 is a pleiotropic developmental regulator required for the maintenance of FLOWERING LOCUS C expression and repression of flowering in Arabidopsis. Plant Cell 17, 2633-2646.
Deal, R. B., Topp, C. N., McKinney, E. C., and Meagher, R. B. (2007). Repression of flowering in Arabidopsis requires activation of FLOWERING LOCUS C expression by the histone variant H2A. Z. Plant Cell 19, 74-83.
Fan, J. Y., Gordon, F., Luger, K., Hansen, J. C., and Tremethick, D. J. (2002). The essential histone variant H2A.Z regulates the equilibrium between different chromatin conformational states. Nat Struct Biol 9, 172-176.
Fitter, A. H., and Fitter, R. S. (2002). Rapid changes in flowering time in British plants. Science 296, 1689-1691.
Gasch, A. P., Spellman, P. T., Kao, C. M., Carmel-Harel, O., Eisen, M. B., Storz, G., Botstein, D., and Brown, P. O. (2000). Genomic expression programs in the response of yeast cells to environmental changes. Mol Biol Cell 11, 4241-4257.
Gendrel, A. V., Lippman, Z., Yordan, C., Colot, V., and Martienssen, R. A. (2002). Dependence of heterochromatic histone H3 methylation patterns on the Arabidopsis gene DDM1. Science 297, 1871-1873.
Gray, W. M., Ostin, A., Sandberg, G., Romano, C. P., and Estelle, M. (1998). High temperature promotes auxin-mediated hypocotyl elongation in Arabidopsis. Proc Natl Acad Sci USA 95, 7197-7202.
Halliday, K. J., Salter, M. G., Thingnaes, E., and Whitelam, G. C. (2003). Phytochrome control of flowering is temperature sensitive and correlates with expression of the floral integrator FT. Plant J 33, 875-885.
Heggie, L., and Halliday, K. J. (2005). The highs and lows of plant life: temperature and light interactions in development. Int J Dev Biol 49, 675-687.
Hodges, C., Bintu, L., Lubkowska, L., Kashlev, M., and Bustamante, C. (2009). Nucleosomal fluctuations govern the transcription dynamics of RNA polymerase II. Science 325, 626-628.
Ishibashi, T., Dryhurst, D., Rose, K. L., Shabanowitz, J., Hunt, D. F., and Ausio, J. (2009). Acetylation of vertebrate H2A.Z and its effect on the structure of the nucleosome. Biochemistry 48, 5007-5017.
Kaplan, N., Moore, I. K., Fondufe-Mittendorf, Y., Gossett, A. J., Tillo, D., Field, Y., LeProust, E. M., Hughes, T. R., Lieb, J. D., Widom, J., et al. (2009). The DNA-encoded nucleosome organization of a eukaryotic genome. Nature 458, 362-366.
Kelly, A. E., and Goulden, M. L. (2008). Rapid shifts in plant distribution with recent climate change. Proc Natl Acad Sci USA 105, 11823-11826.
Kim, H. J., Hyun, Y., Park, J. Y., Park, M. J., Park, M. K., Kim, M. D., Lee, M. H., Moon, J., Lee, I., and Kim, J. (2004). A genetic link between cold responses and flowering time through FVE in Arabidopsis thaliana. Nat Genet 36, 167-171.
Kluth, A., Sprunck, S., Becker, D., Lorz, H., and Lutticke, S. (2002). 5′ deletion of a gbss1 promoter region from wheat leads to changes in tissue and developmental specificities. Plant Mol Biol 49, 669-682.
Knezetic, J. A., and Luse, D. S. (1986). The presence of nucleosomes on a DNA template prevents initiation by RNA polymerase II in vitro. Cell 45, 95-104.
Kobor, M. S., Venkatasubrahmanyam, S., Meneghini, M. D., Gin, J. W., Jennings, J. L., Link, A. J., Madhani, H. D., and Rine, J. (2004). A protein complex containing the conserved Swi2/Snf2-related ATPase Swr1p deposits histone variant H2A.Z into euchromatin. PLoS Biol 2, E131.
Koini, M. A., Alvey, L., Allen, T., Tilley, C. A., Harberd, N. P., Whitelam, G. C., and Franklin, K. A. (2009). High temperature-mediated adaptations in plant architecture require the bHLH transcription factor PIF4. Curr Biol 19, 408-413.
Kovalchuk, N., Smith, J., Pallotta, M., Singh, R., Ismagul, A., Eliby, S., Bazanova, N., Milligan, A. S., Hrmova, M., Langridge, P., et al. (2009). Characterization of the wheat endosperm transfer cell-specific protein TaPR60. Plant Mol Biol 71, 81-98.
Krogan, N. J., Keogh, M. C., Datta, N., Sawa, C., Ryan, O. W., Ding, H., Haw, R. A., Pootoolal, J., Tong, A., Canadien, V., et al. (2003). A Snf2 family ATPase complex required for recruitment of the histone H2A variant Htz1. Mol Cell 12, 1565-1576.
Lam, F. H., Steger, D. J., and O'Shea, E. K. (2008). Chromatin decouples promoter threshold from dynamic range. Nature 453, 246-250.
Larkindale, J., and Vierling, E. (2008). Core genome responses involved in acclimation to high temperature. Plant Physiol 146, 748-761.
Lee, J. H., Yoo, S. J., Park, S. H., Hwang, I., Lee, J. S., and Ahn, J. H. (2007). Role of SVP in the control of flowering time by ambient temperature in Arabidopsis. Genes Dev 21, 397-402.
Lenoir, J., Gegout, J. C., Marquet, P. A., de Ruffray, P., and Brisse, H. (2008). A significant upward shift in plant species optimum elevation during the 20th century. Science 320, 1768-1771.
Li, B., Pattenden, S. G., Lee, D., Gutierrez, J., Chen, J., Seidel, C., Gerton, J., and Workman, J. L. (2005). Preferential occupancy of histone variant H2AZ at inactive promoters influences local histone modifications and chromatin remodeling. Proc Natl Acad Sci USA 102, 18385-18390.
Li, M., Singh, R., Bazanova, N., Milligan, A. S., Shirley, N., Langridge, P., and Lopato, S. (2008). Spatial and temporal expression of endosperm transfer cell-specific promoters in transgenic rice and barley. Plant Biotechnol J 6, 465-476.
Lorch, Y., LaPointe, J. W., and Kornberg, R. D. (1987). Nucleosomes inhibit the initiation of transcription but allow chain elongation with the displacement of histones. Cell 49, 203-210.
Martin-Trillo, M., Lazaro, A., Poethig, R. S., Gomez-Mena, C., Pineiro, M. A., Martinez-Zapater, J. M., and Jarillo, J. A. (2006). EARLY IN SHORT DAYS 1 (ESD1) encodes ACTIN-RELATED PROTEIN 6 (AtARP6), a putative component of chromatin remodelling complexes that positively regulates FLC accumulation in Arabidopsis. Development 133, 1241-1252.
Mavrich, T. N., Jiang, C., Ioshikhes, I. P., Li, X., Venters, B. J., Zanton, S. J., Tomsho, L. P., Qi, J., Glaser, R. L., Schuster, S. C., et al. (2008). Nucleosome organization in the Drosophila genome. Nature 453, 358-362.
Meneghini, M. D., Wu, M., and Madhani, H. D. (2003). Conserved histone variant H2A.Z protects euchromatin from the ectopic spread of silent heterochromatin. Cell 112, 725-736.
Michael, T. P., Mockler, T. C., Breton, G., McEntee, C., Byer, A., Trout, J. D., Hazen, S. P., Shen, R., Priest, H. D., Sullivan, C. M., et al. (2008). Network discovery pipeline elucidates conserved time-of-day-specific cis-regulatory modules. PLoS Genet 4, e14.
Mittler, R. (2006). Abiotic stress, the field environment and stress combination. Trends Plant Sci 11, 15-19.
Mizuguchi, G., Shen, X., Landry, J., Wu, W. H., Sen, S., and Wu, C. (2004). ATP-driven exchange of histone H2AZ variant catalyzed by SWR1 chromatin remodeling complex. Science 303, 343-348.
Nguyen, V. Q., and Sil, A. (2008). Temperature-induced switch to the pathogenic yeast form of Histoplasma capsulatum requires Ryp1, a conserved transcriptional regulator. Proc Natl Acad Sci USA 105, 4880-4885.
Petesch, S. J., and Lis, J. T. (2008). Rapid, transcription-independent loss of nucleosomes over a large chromatin domain at Hsp70 loci. Cell 134, 74-84.
Poethig, R. S. (2003). Phase change and the regulation of developmental timing in plants. Science 301, 334-336.
Polach, K. J., and Widom, J. (1995). Mechanism of protein access to specific DNA sequences in chromatin: a dynamic equilibrium model for gene regulation. J Mol Biol 254, 130-149.
Raisner, R. M., Hartley, P. D., Meneghini, M. D., Bao, M. Z., Liu, C. L., Schreiber, S. L., Rando, O. J., and Madhani, H. D. (2005). Histone variant H2A.Z marks the 5′ ends of both active and inactive genes in euchromatin. Cell 123, 233-248.
Salome, P. A., and McClung, C. R. (2005). PSEUDO-RESPONSE REGULATOR 7 and 9 are partially redundant genes essential for the temperature responsiveness of the Arabidopsis circadian clock. Plant Cell 17, 791-803.
Samach, A., and Wigge, P. A. (2005). Ambient temperature perception in plants. Curr Opin Plant Biol 8, 483-486.
Segal, E., and Widom, J. (2009). From DNA sequence to transcriptional behaviour: a quantitative approach. Nat Rev Genet 10, 443-456.
Sung, D. Y., Vierling, E., and Guy, C. L. (2001). Comprehensive expression profile analysis of the Arabidopsis Hsp70 gene family. Plant Physiol 126, 789-800.
Sung, S., and Amasino, R. M. (2005). Remembering winter: toward a molecular understanding of vernalization. Annu Rev Plant Biol 56, 491-508.
Thambirajah, A. A., Dryhurst, D., Ishibashi, T., Li, A., Maffey, A. H., and Ausio, J. (2006). H2A.Z stabilizes chromatin in a way that is dependent on core histone acetylation. J Biol Chem 281, 20036-20044.
Till, B. J., Zerr, T., Comai, L., and Henikoff, S. (2006). A protocol for TILLING and Ecotilling in plants and animals. Nat Protoc 1, 2465-2477.
Tsankov, A. M., Brown, C. R., Yu, M. C., Win, M. Z., Silver, P. A., and Casolari, J. M. (2006). Communication between levels of transcriptional control improves robustness and adaptivity. Molecular systems biology 2, 65.
Whittle, C. M., McClinic, K. N., Ercan, S., Zhang, X., Green, R. D., Kelly, W. G., and Lieb, J. D. (2008). The genomic distribution and function of histone variant HTZ-1 during C. elegans embryogenesis. PLoS Genet 4, e1000187.
Wilczek, A. M., Roe, J. L., Knapp, M. C., Cooper, M. D., Lopez-Gallego, C., Martin, L. J., Muir, C. D., Sim, S., Walker, A., Anderson, J., et al. (2009). Effects of Genetic Perturbation on Seasonal Life History Plasticity. Science 323, 930-934.
Willis, C. G., Ruhfel, B., Primack, R. B., Miller-Rushing, A. J., and Davis, C. C. (2008). Phylogenetic patterns of species loss in Thoreau's woods are driven by climate change. Proc Natl Acad Sci USA 105, 17029-17033.
Zhang, H., Roberts, D. N., and Cairns, B. R. (2005). Genome-wide dynamics of Htz1, a histone H2A variant that poises repressed/basal promoters for activation through histone loss. Cell 123, 219-231.
Zilberman, D., Coleman-Derr, D., Ballinger, T., and Henikoff, S. (2008). Histone H2A.Z and DNA methylation are mutually antagonistic chromatin marks. Nature 456, 125-129.

1. A method for altering temperature sensing in a eukaryotic organism, the method comprising modifying the presence or state of H2A.Z in a nucleosome of the organism.

2. The method of claim 1, wherein the organism is selected from the group consisting of plants, yeast, fungi, algae, invertebrates and vertebrates.

3. The method of claim 2, wherein the organism is a plant or a part thereof.

4. (canceled)

5. The method of claim 3, wherein the plant is a crop plant.

6. The method of claim 5, wherein the crop plant is selected from the group consisting of rice, wheat, barley, soya, brassica, corn, rye, sorghum and sugarcane.

7. The method of claim 1, wherein the method inhibits the response of the organism to an increase in ambient temperature, and the presence or state of H2A.Z in the nucleosome is modified by increasing the level of H2A.Z in the nucleosome, altering the post-translational modification of H2A.Z such that the H2A.Z is less acetylated, or inhibiting disassembly of the H2A.Z from the nucleosome.

8. The method of claim 1, wherein the method inhibits the response of the organism to a decrease in ambient temperature, and the presence or state of H2A.Z in the nucleosome is modified by decreasing the level of H2A.Z in the nucleosome or decreasing or inhibiting the expression of one or more nucleic acid sequences encoding H2A.Z.

9. The method of claim 8, wherein the nucleic acid sequences are decreased or inhibited using RNA interference.

10. The method of claim 7, wherein the state of H2A.Z in the nucleosome is modified by altering the post-translational modification of H2A.Z by:
a) producing, in the organism, a mutant H2A.Z protein having no acetylation, sites or a reduced number of acetylation sites;
b) increasing the expression of a nucleic acid sequence encoding a histone deacetylase (HDAC) enzyme or increasing the activity of HDAC;
c) decreasing the expression of a nucleic acid sequence encoding a histone acetyltransferase (HAT) enzyme or decreasing the activity of HAT;
or combinations thereof.

11.-34. (canceled)

35. The method of claim 1, wherein the organism is a rice plant or wheat plant and wherein the method enhances grain-fill, grain composition or grain quantity; or prevents premature flowering or bolting at temperatures higher than those at which grain-fill typically occurs efficiently, or at which grain composition and quantity is typically perturbed or at which premature flowering or bolting typically occurs.

36. A plant H2A.Z molecule in which some or all of the lysine residues of the molecule have been replaced with non-acetylatable amino acid residues.

37. A plant comprising the H2A.Z molecule of claim 36 or a nucleic acid sequence encoding the H2A.Z molecule.

38. A plant comprising:
a) a non-acetylatable version of histone H2A.Z;
b) an over-expressed histone deacetylase enzyme sufficient to remove histone acetylation marks in H2A.Z; or
c) a down regulated histone acetyltransferase activity in said organism; or combinations thereof.

39. The plant H2A.Z molecule of claim 36, wherein the plant is selected from the group consisting of moncots and dicots.

40. The plant H2A.Z molecule of claim 39, wherein the plant is a crop plant.

41. The plant H2A.Z molecule of claim 40, wherein the crop plant is selected from the group consisting of rice, wheat, barley, soya, brassica, corn, rye, sorghum and sugarcane.

42. A method for inhibiting pathogenesis of a eukaryotic micro-organism, the method comprising modifying the presence of H2A.Z in a nucleosome of the eukaryotic microorganism, wherein modification perturbs temperature perception in the eukaryotic microorganism and therefore inhibits the signal inducing pathogenesis.
